(19) United States
(12) Patent Application Publication
(10) Pub. No.: US 2026/0062464 A1
(43) Pub. Date: Mar. 5, 2026

(54) MONOCLONAL ANTIBODIES THAT BIND TO THE UNDERSIDE OF INFLUENZA VIRAL NEURAMINIDASE

(71) Applicant: The U.S.A., as represented by the Secretary, Department of Health and Human Services, Bethesda, MD (US)

(72) Inventors: Masaru Kanekiyo, Chevy Chase, MD (US); Julia Lederhofer, Washington, DC (US); Yaroslav Tsybovsky, Brunswick, MD (US); Sarah F. Andrews, Bethesda, MD (US)

(73) Assignee: THE U.S.A., as represented by the Secretary, Department of Health and Human Services, Bethesda, MD (US)

(21) Appl. No.: 19/100,498

(22) PCT Filed: Jul. 28, 2023
(86) PCT No.: PCT/US2023/071194

§ 371 Date: Jan. 31, 2025

Related U.S. Application Data
(60) Provisional application No. 63/394,243, filed on Aug. 1, 2022.

(51) Int. Cl. C07K 16/10; A61K 39/00; A61P 31/16

(52) U.S. Cl. CPC C07K 16/108; A61P 31/16; A61K 2039/505

(57) ABSTRACT

Monoclonal antibodies are antigen binding fragments are disclosed that specifically bind to the underside of influenza A neuraminidase (NA). In some aspects, these antibodies and antigen binding fragments are used for in methods of treating a subject with an influenza A infection, such as an H3N2 infection. In more aspects, these antibodies and antigen binding fragments and bispecific antibodies for detection of an influenza virus in a sample, and for selecting vaccines.

### CROSS REFERENCE TO RELATED APPLICATION

This application claims priority to U.S. Provisional Application No. 63/394,243, filed Aug. 1, 2022, which is incorporated herein by reference in its entirety.

### STATEMENT OF GOVERNMENT SUPPORT

This invention was made with Government support under project number Z01 #: HHSN261200800001 by the National Institutes of Health, Frederik National Laboratory for Cancer Research. Additionally, this work was supported, in part, by Intramural Research Program of the Vaccine Research Center, NIADI, NIH. The United States Government has certain rights in the invention.

### FIELD OF THE DISCLOSURE

This relates to monoclonal antibodies and antigen binding fragments that specifically bind to the underside of influenza A neuraminidase (NA) and their use, for example, in methods of treating a subject with an influenza A infection, detecting influenza in a sample, and for selecting vaccines.

### REFERENCE TO AN ELECTRONIC SEQUENCE LISTING

The contents of the electronic sequence listing (Sequences.xml, which has a size of 54,947 bytes and a Date of Creation of Jul. 18, 2023) is herein incorporated by reference in its entirety.

### BACKGROUND

Improved vaccines against seasonal influenza virus are highly needed to provide broader protection against diverse influenza strains. Continuously evolving influenza viruses cause seasonal epidemics and pose global pandemic threats. Although viral neuraminidase (NA) is an effective drug and vaccine target, the understanding of the NA antigenic landscape still remains incomplete. The use of broadly cross-group binding neutralizing monoclonal antibodies that target different epitopes can help to design better immunogens, and can be used therapeutically and prophylactically.

### SUMMARY OF THE DISCLOSURE

Monoclonal antibodies and antigen binding fragments thereof are disclosed herein that specifically bind the underside of NA of an influenza virus. In some aspects, the monoclonal antibodies bind the NA of H3N2 influenza virus.

In some aspects, disclosed is an isolated monoclonal antibody, including a heavy chain variable region and a light chain variable region comprising a heavy chain complementarity determining region (HCDR)1, a HCDR2, and a HCDR3 and a light chain complementarity determining region (LCDR)1, a LCDR2, and a LCDR3 of the V_{H} and V_{L} set forth as one of:
a) SEQ ID NOs: 1 and 5, respectively (NDS.1);
b) SEQ ID NOs: 17 and 21, respectively (NDS.3);
c) SEQ ID NOs: 9 and 13, respectively (NDS.1.1);
d) SEQ ID NOs: 25 and 29, respectively (NDS.1.2);
e) SEQ ID NOs: 33 and 37, respectively (NDS.6);
f) SEQ ID NOs: 41 and 45, respectively (NDS.8);
g) SEQ ID NOs: 49 and 53, respectively (NDS.5); or
h) SEQ ID NOs: 57 and 61, respectively (NDS.7);
   wherein the monoclonal antibody specifically binds to influenza A neuraminidase (NA) protein and inhibit a H3N2 influenza A virus.

In some aspects, methods are provided for preventing or treating influenza in a subject. These methods include administering to the subject a therapeutically effective amount of a disclosed antibody or antigen binding fragment, or a nucleic acid encoding the antibody or the antigen binding fragment. In some aspects, the influenza virus can be an H3N2 influenza virus.

In additional aspects, methods are provided for detecting an influenza virus infection in a subject. The methods include contacting a biological sample from the subject with a disclosed monoclonal antibody or the antigen binding fragment, and detecting antibody bound to the sample. The presence of antibody bound to the sample indicates that the subject has an influenza virus infection. In some aspects, the influenza virus can be an H3N2 influenza virus.

In other aspects, methods are provided for identifying a protein as a vaccine candidate for preventing or treating an influenza virus infection. The methods include contacting the protein with a disclosed monoclonal antibody or the antigen binding fragment, under conditions sufficient to form an immune complex, and detecting the presence of an immune complex. The presence of the immune complex indicates that the protein is a vaccine candidate for an influenza virus infection. In some aspects, the influenza virus can be an H3N2 influenza virus.

The foregoing and other features of the disclosure will become more apparent from the following detailed description of several aspects which proceeds with reference to the accompanying figures.

### BRIEF DESCRIPTION OF THE FIGURES

FIGS. 1A-1D: Identification and characterization of pan-N2 NA-targeting antibodies. FIG. 1A: Immunogenetics of N2-specific B cells. Immunoglobulin variable heavy and light chain gene usage and V-D-J junction of NA-specific B cells for two donors are shown. Each pie slice indicates a B cell clone with the same V_{H} and V_{K}/V_{L} gene usage and similar CDR H3 identity. The total number of paired heavy and light chain sequences analyzed is shown inside each pie chart. Immunoglobulin gene usage and CDR H3 sequences of dark side mAbs we characterized are shown, with the corresponding SEQ ID NO indicated. FIG. 1B: ELISA binding curves of dark side mAbs against recombinant N2 protein of A/Moscow/10/1999 (H3N2, MO99); A/Wisconsin/67/2005 (WI05, H3N2); A/Darwin/09/2021 (DW21, H3N2); and A/Indiana/10/2011 (IN11v, H3N2v). FIG. 1C: Cross-competition profile of NA-targeting mAbs, shown as a heatmap. Binding was measured by BLI with recombinant WI05 N2 protein. FIG. 1D: Binding affinity of Fab of dark side mAbs to recombinant N2 NAs. Binding kinetics of Fabs were measured by BLI with recombinant MO99, WI05, DW21, and IN11v NAs. K_{D} values calculated using the Langmuir 1:1 interaction model for each Fab-NA pair are shown.

FIGS. 2A-2E: Cryo-EM structure of NDS.1 and 1G01 Fabs in complex with IN11v N2 NA. FIG. 2A: Consensus cryo-EM map of NA Indiana N2 with bound NDS.1 and 1G01 Fabs, with docked atomic models of NA and 1G01, respectively. Glycans are shown in stick representation. The cryo-EM density for NDS.1 is weaker due to partial occupancy. FIG. 2B: Local 3D classification and refinement of a region containing one NA protomer with two bound Fabs. The heavy and light chains of NDS.1 are shown. FIG. 2C: Ribbon diagram of the NA-NDS.1 complex. Only the heavy chain of NDS.1 interacts with NA. The six four-stranded β sheets forming the structure of the NA monomer are labeled with Roman numerals. FIG. 2D: Contact interface between NA and NDS.1. NA Indiana N2 is shown in surface representation. The CDRs and FRs of the NDS.1 heavy chain participating in the interaction are depicted as ribbons, with residues in contact with NA shown in stick representation and labeled. FIG. 2E: Detailed illustration of the interactions between NA and NDS.1. Only the molecular regions participating in the interaction are shown for clarity. Residues forming hydrogen bonds are shown in stick representation and labeled for NA and NDS.1, respectively. Dashed lines depict hydrogen bonds.

FIGS. 3A-3E: Cryo-EM structure of NDS.3 Fab in complex with DW21 N2 NA. FIG. 3A: Cryo-EM map and docked atomic models of NA Darwin N2 in complex with NDS.3 Fab. The heavy and light chains of NDS.3 are shown. Glycans are shown in stick representation. FIG. 3B: Ribbon diagram of the NA-NDS.3 complex. FIG. 3C: Fab NDS.3 interacts primarily with β sheet III of NA. NA orientation is the same as in FIG. 2C, right panel. FIG. 3D: Contact interface between NA and NDS.3. NA Darwin N2 is shown in surface representation. The CDRs of Fab NDS.3 are depicted as ribbon and labeled, with residues in contact with NA shown as sticks and labeled. Asterisks denote NDS.3 residues contacting both NA protomers. FIG. 3E: Detailed illustration of the interactions between protomer 1 of NA and Fab NDS.3. Only the molecular regions participating in the interaction are shown for clarity. Residues forming hydrogen bonds and salt bridges are shown in stick representation for NA and NDS.3. Dashed lines depict hydrogen bonds and salt bridges.

FIGS. 4A-4E: NA underside epitopes recognized by NDS.1 and NDS.3. FIG. 4A: Surface representation of the NA head tetramer with the epitopes of NDS.1 and NDS.3. Dots indicate the positions of the NA residues that contribute to both epitopes. Epitopes are shown only on one NA protomer for clarity. FIG. 41B: Ribbon diagram showing Fabs of NDS.1 and NDS.3 interacting with NA tetramer). Bound Fabs are shown only on one NA protomer for clarity. FIG. 4C: NA residues contributing to both NDS.1 and NDS.3 epitopes. NA, NDS.3 heavy chain, and NDS.1 light chain are shown. Dashed lines depict hydrogen bonds. FIG. 4D: Sequence alignment of NAs of IN11v (SEQ ID NO: 65) and DW21 (SEQ ID NO: 66). Residues forming the epitopes of 1G01, NDS.1, and NDS.3 are shown. The residues at positions 1-82 are part of the transmembrane and stalk region and were not included in the recombinant soluble NA tetramers used for structural studies. FIG. 4E: Sequence conservation of NDS.1 and NDS.3 epitopes among NAs of human H3N2 and HxN2. NDS.1 and NDS.3 contact residues are indicated above the sequence logos plots. Open circles denote contacts formed by NA main-chain atoms only; open circles with rays denote side-chain contacts only; filled circles denote both main- and side-chain contacts. Light grey residues are not part of the epitopes.

FIGS. 5A-5C: In vitro virus inhibition by NA dark side-directed mAbs. FIG. 5A: NAI activity of the mAbs against H3N2 viruses measured by IRINA. FIG. 5B: NAI activity of the mAbs against H3N2 viruses measured by ELLA. FIG. 5C: Viral growth inhibition of the mAbs against H3N2, H3N2v, and H2N2 viruses. The half-maximal inhibitory concentration (IC₅₀) of mAb for each virus was calculated from two independent experiments and mean IC₅₀ values for each mAb-virus combination are shown.

FIGS. 6A-6B: Protective efficacy of the NA dark side-directed mAbs against A/Philippines/2/1982 (H3N2). FIG. 6A: Pre-exposure prophylaxis experiment. BALB/c mice were treated with 10, 3, or 1 mg kg⁻¹ of mAbs intraperitoneally 24 h prior to the intranasal virus challenge. Virus dose was 10×LD₅₀. Group size was 10 except for NDS.1 at 1 mg kg⁻¹ (n=9). Percent survival (upper panels) and percent body weight change (lower panels) are shown. FIG. 6B: Post-exposure treatment experiment. BALB/c mice were infected with virus prior to mAb administration (10 mg kg⁻¹) at 48 h post-infection. Virus dose was 5×LD₅₀. Group size was 10 except for NDS.1 group (n=9). Percent survival (upper) and percent body weight change are shown.

FIGS. 7A-7C: Flow cytometry gating strategy and antibody binding kinetics, Related to FIGS. 1A-1D. FIG. 7A: Example of flow cytometry gating strategy for IgG⁺ positive B cells of donor A. FIG. 7B: Flow plots of NA-specific memory IgG⁺ B cells of donor A and B. Gating was used for sorting. Donor A (top panel) was sorted with N2 WI05 using two different fluorochromes. Donor B (bottom panel) was sorted with N2 WI05 and N2 DW21 probes. FIG. 7C: Binding sensorgrams of NDS.1, NDS.1.1 and NDS.3 Fabs binding to A/Moscow/10/1999 (H3N2, MO99); A/Wisconsin/67/2005 (WI05, H3N2); A/Darwin/09/2021 (DW21, H3N2); and A/Indiana/10/2011 (IN11v, H3N2v). Binding kinetics of NDS.1 Fab to WI05 and DW21. Experimental data were fitted with the binding equations describing a 1:1 interaction.

FIG. 8: Cryo-EM data processing workflow for NA Indiana N2 in complex with Fabs NDS.1 and 1G01, data related to FIGS. 2A-2E.

FIGS. 9A-9F: Validation of the consensus cryoEM map of NA Indiana N2 in complex with Fabs NDS.1 and 1G01, Related to FIGS. 2A-2E. FIG. 9A: Representative micrograph. FIG. 9B: Representative high-resolution 2D class averages. FIG. 9C: Gold-standard resolution data generated by Relion. At the 0.143 threshold, the resolution is 3.0 Å. FIG. 9D: Fourier shell correlation curve between the map and the atomic model. FIG. 9E: Results of local resolution analysis using ResMap. The cryo-EM map is presented according to local resolution. FIG. 9F: Examples of cryo-EM density.

FIGS. 10A-10D: Validation of the local cryoEM map of NA Indiana N2 in complex with Fabs NDS.1 and 1G01, Related to FIGS. 2A-2E. FIG. 10A: Gold-standard resolution data generated by Relion. At the 0.143 threshold, the resolution is 3.6 Å. FIG. 10B: Fourier shell correlation curve between the map and the atomic model. FIG. 10C: Results of local resolution analysis using ResMap. The cryo-EM map is colored according to local resolution. FIG. 10D: Examples of cryo-EM density.

FIG. 11: Cryo-EM data processing workflow for NA Darwin N2 in complex with Fab NDS.3, data related to FIGS. 3A-3E.

FIGS. 12A-12F: Validation of the cryoEM map of NA Darwin N2 in complex with Fab NDS.3, Related to FIGS. 3A-3E. FIG. 12A: Representative micrograph. FIG. 12B: Representative high-resolution 2D class averages. FIG. 12C: Gold-standard resolution data generated by Relion. At the 0.143 threshold, the resolution is 2.7 Å. FIG. 12D: Fourier shell correlation curve between the map and the atomic model. FIG. 12E: Results of local resolution analysis using ResMap. The cryo-EM map is colored according to local resolution. FIG. 12F: Examples of cryo-EM density.

FIG. 13: Table of binding affinity of NDS.1, NDS.1.1 and NDS.3 Fabs to N2 NAs.

FIG. 14: Table providing a summary of mAb binding to group 1 and group 2 NAs measured by BLI.

FIG. 15: Table providing cryo-EM data collection and single particles analysis statistics.

FIG. 16: Table providing the statistical analysis of Kaplan-Meier curves.

FIG. 17 Characterization of pan-N2 NA-targeting antibodies. This figure shows cross competition of binding of the underside antibodies to another monoclonal antibody that binds to the NA catalytic site (1G01). This experiment was done by using Octet. The biosensors were coated with N2 NA (Wisconsin 2005) and dipped in the first antibody followed by the second antibody. The darker the color the more inhibition is present which means that the second antibody binds to the same, partially overlapping, or proximal epitope as the first antibody. The NA catalytic site-directed antibody 1G01 (Stadlbauer et al, Science 2019) is used as a control, showing no cross competition with the underside antibodies.

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.

SEQ ID NO: 1 is the amino acid sequence of the heavy chain variable (V_{H}) domain of NDS.1.1.

SEQ ID NOs: 2, 3 and 4 are the amino acid sequence of the IMGT (IMMUNOGENETICS® information system) HCDR1, HCDR2, and HCDR3 of NDS.1.1.

SEQ ID NO: 5 is the is the amino acid sequence of the light chain variable (V₁.) domain of NDS.1.1.

SEQ ID NOs: 6, 7 and 8 are the amino acid sequence of the IMGT LCDR1, LCDR2, and LCDR3 of NDS.1.1.

SEQ ID NO: 9 is the amino acid sequence of the V_{H} domain of NDS.1.

SEQ ID NOs: 10, 11 and 12 are the amino acid sequence of the IMGT HCDR1, HCDR2, and HCDR3 of NDS.1.

SEQ ID NO: 13 is the is the amino acid sequence of the V_{L} domain of NDS.1.

SEQ ID NOs: 14, 15 and 16 are the amino acid sequence of the IMGT LCDR1, LCDR2, and LCDR3 of NDS.1.

SEQ ID NO: 17 is the amino acid sequence of the V_{H} domain of NDS.3.

SEQ ID NOs: 18, 19 and 20 are the amino acid sequence of the IMGT HCDR1, HCDR2, and HCDR3 of NDS.3.

SEQ ID NO: 21 is the is the amino acid sequence of the V_{L} domain of NDS.3.

SEQ ID NOs: 22, 23 and 24 are the amino acid sequence of the IMGT LCDR1, LCDR2, and LCDR2 of NDS.3.

SEQ ID NO: 25 is the amino acid sequence of the V_{H} domain of NDS.1.2.

SEQ ID NOs: 26, 27, and 28 are the amino acid sequence of the IMGT HCDR1, HCDR2, and HCDR3 of NDS.1.2.

SEQ ID NO: 29 is the is the amino acid sequence of the V_{L} domain of NDS.1.2.

SEQ ID NOs: 30, 31 and 32 are the amino acid sequence of the IMGT LCDR1, LCDR2, and LCDR2 of NDS.1.2.

SEQ ID NO: 33 is the amino acid sequence of the V_{H} domain of NDS.6.

SEQ ID NOs: 34, 35 and 36 are the amino acid sequence of the IMGT HCDR1, HCDR2, and HCDR3 of NDS.6.

SEQ ID NO: 37 is the is the amino acid sequence of the V_{L} domain of NDS.6.

SEQ ID NOs: 38, 39 and 40 are the amino acid sequence of the IMGT LCDR1, LCDR2, and LCDR2 of NDS.6.

SEQ ID NO: 41 is the amino acid sequence of the V_{H} domain of NDS.8.

SEQ ID NOs: 42, 43 and 44 are the amino acid sequence of the IMGT HCDR1, HCDR2, and HCDR3 of NDS.8.

SEQ ID NO: 45 is the is the amino acid sequence of the V_{L} domain of NDS.8.

SEQ ID NOs: 46, 47 and 48 are the amino acid sequence of the IMGT LCDR1, LCDR2, and LCDR2 of NDS.8.

SEQ ID NO: 49 is the amino acid sequence of the V_{H} domain of NDS.5.

SEQ ID NOs: 50, 51 and 52 are the amino acid sequence of the IMGT HCDR1, HCDR2, and HCDR3 of NDS.5.

SEQ ID NO: 53 is the is the amino acid sequence of the V_{L} domain of NDS.5.

SEQ ID NOs: 54, 55 and 56 are the amino acid sequence of the IMGT LCDR1, LCDR2, and LCDR3 of NDS.5.

SEQ ID NO: 57 is the amino acid sequence of the V_{H} domain of NDS.7.

SEQ ID NOs: 58, 59 and 60 are the amino acid sequence of the IMGT HCDR1, HCDR2, and HCDR3 of NDS.7.

SEQ ID NO: 61 is the is the amino acid sequence of the V_{L} domain of NDS.7.

SEQ ID NOs: 62, 63, and 64 are the amino acid sequence of the IMGT LCDR1, LCDR2, and LCDR3 of NDS.7.

SEQ ID NOs: 65 and 66 are the amino acid sequences of IN11v and DW21 neuraminidase (NA), respectively.

### DETAILED DESCRIPTION OF SEVERAL ASPECTS

Disclosed herein are monoclonal antibodies and antigen binding fragments that specifically bind to the underside of influenza A NA and their use, for example, in methods of treating a subject with an influenza A infection, such as an H3N2 infection, and their use for detection of an influenza virus in a sample, and for selecting vaccines.

A group of mAbs were isolated that bind to N2 and neutralize influenza A. These antibodies inhibit propagation in vitro of influenza A H3N2 and H2N2 virus. Additionally, these antibodies confer protection against H2N2 infection in mice in both prophylactic and therapeutic settings. 3D class images taken with cryo-EM show that several of the mAbs target conserved epitopes on the underside of the globular head of N2 neuraminidases (NA).

Antibodies described in literature bind to the enzymatic active site or the side of NA and not to the underside of NA. The disclosed results show that the head of NA must be flexible since these monoclonal antibodies were isolated after infection. This discovery can be useful for vaccine design strategies. Moreover, the monoclonal antibodies can be used as a targeted therapy against influenza disease.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as antibody) is administered by introducing the composition into a blood vessel of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody and Antigen Binding Fragment: An immunoglobulin, antigen-binding fragment, or derivative thereof, that specifically binds and recognizes an analyte (antigen) such as a NA protein, such as the underside of an NA protein. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, TgD, TgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NTH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some aspects, a disclosed antibody includes a heterologous constant domain. For example, the antibody can include a constant domain that is different from a native constant domain, such as a constant domain including one or more modifications (such as the “LS” mutation) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one aspect, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some aspects, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1St Ed. New York: Cold Spring Harbor Laboratory Press, 2004. Print; Lonberg, Nat. Biotech., 23: 1117-1125, 2005; Lonenberg, Curr. Opin. Immunol., 20:450-459, 2008).

Antibody or antigen binding fragment that neutralizes an influenza virus: An antibody or antigen binding fragment that specifically binds to an influenza antigen (such as the NA protein) in such a way as to inhibit a biological function associated with the influenza virus that inhibits infection. The antibody can neutralize the activity of one or more influenza viruses. For example, an antibody or antigen binding fragment that neutralizes the influenza virus, such as H3N2 may interfere with the virus by binding it directly and limiting entry into cells. Alternately, an antibody may interfere with one or more post-attachment interactions of the pathogen with a receptor, for example, by interfering with viral entry using the receptor. In some examples, an antibody that is specific for an influenza virus neutralizes the infectious titer of this virus.

In some aspects, an antibody or antigen binding fragment that specifically binds to an influenza virus, and neutralizes the influenza virus, inhibits infection of cells, for example, by at least 50% compared to a control antibody or antigen binding fragment.

Biological sample: A sample obtained from a subject. Biological samples include all clinical samples useful for detection of disease or infection in subjects, including, but not limited to, cells, tissues, and bodily fluids, such as blood, sputum, nasal swab, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a influenza virus infection, such as, but not limited to, an influenza virus infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{th} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one aspect, an antibody is linked to an effector molecule; for example, an antibody that specifically binds to the underside of NA, covalently linked to an effector molecule, such as a detectable label. The linkage can be by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a NA-specific antibody can include up to 1, 2, 3, 4, 5, 6, 7, 8, 9, or up to 10 conservative substitutions compared to a reference antibody sequence and retain specific binding activity for spike protein binding, and/or neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to the underside of NA. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some aspects, the control is a negative control, such as sample obtained from a healthy patient not infected with an influenza virus. In other aspects, the control is a positive control, such as a tissue sample obtained from a patient diagnosed with an influenza virus infection. In still other aspects, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effective amount: A quantity of a specific substance sufficient to achieve a desired effect in a subject to whom the substance is administered. For instance, this can be the amount necessary to inhibit an influenza infection, such as an H3N2 or H2N2 influenza virus infection, or to measurably alter outward symptoms of such an infection.

In one example, a desired response is to inhibit or reduce or prevent an influenza virus. The influenza virus infection does not need to be completely eliminated or reduced or prevented for the method to be effective. For example, administration of an effective amount can decrease the influenza virus infection (for example, as measured by infection of cells, or by number or percentage of subjects infected by the influenza virus), for example by at least 50%, at least 60%, at least 70%, at least 80%, at least 90%, at least 95%, at least 98%, or even at least 100% (elimination or prevention of detectable influenza virus infection), as compared to a suitable control.

In some aspects, administration of an effective amount of a disclosed antibody or antigen binding fragment that binds to the underside of NA can reduce or inhibit infection (for example, as measured by infection of cells, or by number or percentage of subjects infected by the influenza virus or by an increase in the survival time of infected subjects, or reduction in symptoms associated with the infection), for example by at least 10%, at least 20%, at least 50%, at least 60%, at least 70%, at least 80%, at least 90%, at least 95%, at least 98%, or even at least 100% (elimination or prevention of detectable infection), as compared to a suitable control.

The effective amount of an antibody or antigen binding fragment that specifically binds the NA that is administered to a subject to inhibit infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes, optionally in combination with previous or subsequent administrations, to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on the underside of NA.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” includes, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to EU numbering. For IgA, the Fc region includes immunoglobulin domains Cα2 and Cα3 and optionally the lower part of the hinge between Cal and Cα2.

Hemagglutinin (HA): An influenza virus surface glycoprotein that is a homotrimeric integral membrane glycoprotein. HA mediates binding of the virus particle to a host cell and subsequent entry of the virus into the host cell. The nucleotide and amino acid sequences of numerous influenza HA proteins are known in the art and are publicly available, such as through the NCBI Influenza Virus Resource database (Bao et al., J Virol 82:596-601, 2008). HA (along with NA) is one of the two major influenza virus antigenic determinants. The three identical monomers that constitute HA are constructed into a central a helix coil; three spherical heads contain the sialic acid binding sites. In nature, HA monomers are synthesized as precursors that are then glycosylated and cleaved into two smaller polypeptides: the HA1 and HA2 subunits. Each HA monomer consists of a long, helical chain anchored in the membrane by HA2 and topped by a large HA1 globular head which contains the sialic acid receptor binding sites. The HA2 protein chain facilitates membrane fusion; the C-terminal end of the protein is embedded in the viral membrane. The stalk of HA is comprised of portions of HA1 and HA2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgA: A polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG₁, IgG₂, IgG₃, and IgG₄.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Influenza virus: A segmented negative-strand RNA virus that belongs to the Orthomyxoviridae family. There are three types of Influenza viruses, A, B and C. Influenza A viruses infect a wide variety of birds and mammals, including humans, horses, marine mammals, pigs, ferrets, and chickens.

Influenza A viruses are categorized into subtypes based on the type of two proteins, hemagglutinin (HA) and neuraminidase (NA) that are on the surface of the viral envelope. Different influenza viruses encode for different HA and NA proteins. There are 18 different HA subtypes and 11 different NA subtypes, H1 through H18 and N1 through N11 respectively.

In animals, most influenza A viruses cause mild localized infections of the respiratory and intestinal tract. However, highly pathogenic influenza A strains, such as H5N1, cause systemic infections in poultry in which mortality may reach 100%. Influenza A viruses that have caused disease in humans include the following subtypes: H1N1, H2N2, and H3N2. Other subtypes of viruses that have infected humans but are not transmitted from person to person are: H5N1 (bird flu), H6N1, H7N7, H1N2, H9N2, H7N2. H7N3, H10N7 and H10N8. There are different lineages of HA and NA within each subtype that are distinguished by amino acid sequence. For example, H1N1 viruses that circulated before 2009 and swine flu H1N1 viruses that have circulated amongst humans since 2009, represent different HA and NA lineages. Within each lineage, virus variants of different “clades” circulate in the population each winter.

Antibodies to NA, particularly those that inhibit its enzyme activity, reduce virus replication because newly formed virus particles cannot be released from the infected cell. Antibodies to HA and NA are associated with resistance to influenza disease.

Inhibiting or treating a disease: Inhibiting the full development of a disease or condition, for example, in a subject who is at risk for a disease such as an influenza virus infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Isolated: A biological component (such as a nucleic acid, peptide, or protein, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation. cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Neuraminidase (NA): An influenza virus membrane glycoprotein. NA is involved in the destruction of the cellular receptor for the viral HA by cleaving terminal sialic acid residues from carbohydrate moieties on the surfaces of infected cells. NA also cleaves sialic acid residues from viral proteins, preventing aggregation of viruses. NA (along with HA) is one of the two major influenza virus antigenic determinants. NA forms a tetramer comprised of four identical subunits (monomers) in vivo. As described under the term “influenza”, antibodies that inhibit NA activity prevent virus spread, thereby limiting the infectious process and reducing morbidity and mortality.

Influenza neuraminidase exists as a mushroom-shape projection on the surface of the influenza virus. It has a head consisting of four co-planar and roughly spherical subunits, and a hydrophobic region that is embedded within the interior of the virus' membrane. The conformation of NAs from H1N1, H3N2 as well as other virus subtypes have been examined by x-ray crystallography. An exemplary GENBANK® accession number for the structure of the NA of A(H1N1)pdm09 virus A/California/07/2009 is 3NSS (Li, Q. et al. Nat Struct Mol Biol 17, 1266-8 (2010), incorporated herein by reference.

The “underside” of NA is the solvent accessible surface located opposite side of the catalytic pocket within the globular head on each NA protomer. In the context of membrane anchored NA tetramer, the underside faces to the membrane where the NA stalk is anchored. Based on the structural domain defined by the crystal structure of N2 NA of A/Tokyo/3/1967 described in Varghee et al. (Nature, 303, 35-40, 1983), the underside surface primarily consists of the following domains: N-terminal strand (positions 84-94); b₄L₁₂ (positions 284-285); b₄L₃₄ (positions 305-306); b₄S₄ (positions 307-314); b₅L₁₂ (positions 357-358); b₅L₃₄ (positions 383-386), and may also contain one or more residues from b₄L₀₁ (positions 269-277); b₅L₀₁ (positions 315-350); b₄S₂ (positions 286-291); b₄S₃ (positions 296-304); and b₅S₁ (positions 351-356).

An antibody that specifically binds an N2 subtype influenza virus does not detectably bind an influenza virus of another subtype, such as N1, N3, N4, etc. An antibody that binds NA can specifically bind to a single variant or all variants within a lineage. The monoclonal antibodies disclosed herein bind to N2 subtype influenza viruses, but does not detectably bind to influenza viruses of subtypes N1, N4, or N5.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{th} ed., London, UK: Pharmaceutical Press, 2013, describes compositions and formulations suitable for pharmaceutical delivery of the disclosed agents.

In general, the nature of the carrier may depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some aspects, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some aspects, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one aspect, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several aspects, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a V_{L}, or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994, Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example the underside of NA and does not bind in a significant amount to other proteins present in the sample or subject. With regard to a NA, the epitope may be present on the spike protein of more than one type of influenza virus, such that the antibody binds to the underside of NA on N2 influenza viruses, but does not bind to other proteins, such as proteins from other viruses or other proteins (non-NA) of influenza virus, such as HA. Specific binding can be determined by standard methods. See Harlow & Lane, Antibodies, A Laboratory Manual, 2^{nd} ed., Cold Spring Harbor Publications, New York (2013), for a description of exemplary immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on NA, such as the underside, can bind molecules/agents including this domain, including viruses, substrate to which the spike protein is attached, or the protein in a biological specimen. It is, of course, recognized that a certain degree of non-specific interaction may occur between an antibody and a non-target. Typically, specific binding results in a much stronger association between the antibody and an NA protein than between the antibody other different influenza proteins (such as the HA protein) or from non-influenza virus proteins. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a bird, such as a chicken on turkey. In an additional example, a subject is selected that is in need of inhibiting an influenza virus infection. For example, the subject is either uninfected and at risk of the influenza virus infection or is infected and in need of treatment.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some aspects, a viral vector comprises a nucleic acid molecule encoding a disclosed antibody or antigen binding fragment that specifically binds to the underside of NA and neutralizes the influenza virus. In some aspects, the viral vector can be an adeno-associated virus (AAV) vector.

Under conditions sufficient for: A phrase that is used to describe any environment that permits a desired activity.

### II. Description of Several Aspects

Isolated monoclonal antibodies and antigen binding fragments that specifically bind the underside of NA are provided. The monoclonal antibodies and antigen binding fragments specifically bind to NA and neutralize influenza virus, such as an N2 influenza virus, for example an H3N2 influenza virus. The antibodies and antigen binding fragments can be fully human. In some aspects the disclosed antibodies can inhibit an influenza virus infection in vivo, and can be administered prior to, or after, an infection with an influenza virus, such as, but not limited to, H3N2. Multispecific antibodies, such as bispecific ispecific antibodies including the variable domains of these antibodies are also provided. In addition, disclosed herein are compositions comprising the antibodies and antigen binding fragments and a pharmaceutically acceptable carrier.

Nucleic acids encoding the antibodies, antigen binding fragments, variable domains, and expression vectors (such as adeno-associated virus (AAV) viral vectors) comprising these nucleic acids are also provided. The antibodies, antigen binding fragments, nucleic acid molecules, host cells, and compositions can be used for research, diagnostic, treatment and prophylactic purposes. For example, the disclosed antibodies and antigen binding fragments can be used to diagnose a subject with an influenza virus infection or can be administered to inhibit an influenza virus infection in a subject.

A. Monoclonal Antibodies that Specifically Bind Influenza NA and Antigen Binding Fragments Thereof

Isolated monoclonal antibodies and antigen binding fragments thereof that specifically bind NA are provided. These antibodies specifically bind the NA of an N2 subtype influenza virus. Thus, the antibodies can be used to detect, or treat, an influenza virus infection such as an N2 influenza virus infection. In some aspects, the antibodies can be used to detect, or treat, an influenza virus infection, such as an H3N2, H2N2, H1N2 and/or an H9N2 influenza virus infection.

The discussion of monoclonal antibodies below refers to isolated monoclonal antibodies that include heavy and/or light chain variable domains (or antigen binding fragments thereof) comprising a CDR1, CDR2, and/or CDR3 with reference to the IMGT numbering scheme (unless the context indicates otherwise). Various CDR numbering schemes (such as the Kabat. Chothia or IMGT numbering schemes) can be used to determine CDR positions. The amino acid sequence and the CDRs of the heavy and light chain of the disclosed monoclonal antibody according to the IMGT numbering scheme are provided in the listing of sequences, but these are exemplary only.

In some aspects, a monoclonal antibody is provided that includes the heavy and light chain CDRs of any one of the antibodies described herein. In some aspect, a monoclonal antibody is provided that includes the heavy and light chain variable regions of any one of the antibodies described herein.

In some aspects, the antibody binds the underside of NA. The amino acid sequences of the VH, VL, HCDRs, and LCDRs are provided in the table below.

TABLE 1
IMGT CDRs of Antibodies and SEQ ID NOs.
Antibody NDS.1.1V_{H}
QVQIQQWGAGLLKPSDTLSLTCSVYGGSFNFYYWTWVRQPPGKGLEWLAEINHSGSINYIP
SLNSRVALSVDTSKGQFSLRLSSVTAADTAVYYCTGVPIDYESLHLGYYPMDVWGQGTPVI
VSS (SEQ ID NO: 1)

| | SEQ ID NO: 1 | | CDR |
|---|---|---|---|
| V_{H} | positions | CDR protein sequence | SEQ ID NO |
| HCDR1 | 76-99 | GGSFNFYY | 2 |
| HCDR2 | 151-171 | INHSGSI | 3 |
| HCDR3 | 286-342 | TGVPIDYESLHLGYYPMDV | 4 |

Antibody NDS.1.1: V_{L}
DIQMTQSPSSLSASVGDRVTITCRASQGISNYLAWWQQKPGQVPKLLIYAASTLQSGVPSRF
SGSGSGTDFSLTISSLQPDDVAIYYCQKYNSAPRTFGQGTKVEIK (SEQ ID NO: 5)

| | SEQ ID NO: 5 | | CDR |
|---|---|---|---|
| VL | positions | CDR Sequence | SEQ ID NO |
| LCDR1 | 77-94 | QGISNY | 6 |
| LCDR2 | 146-154 | AAS | 7 |
| LCDR3 | 263-289 | QKYNSAPRT | 8 |

Antibody NDS.1: V_{H}
QVQLRQWGAGLLKPSETLSLTCDVYGGSFNFYFWTWIRQPPGKGLEWLGEITHSGSATYIPS
LKSRVTLSVDTSKGQFSLKLTSVNVADTAVYYCAGLPIDYDTVHLGYYPMDVWGQGTPVT
VSS (SEQ ID NO: 9)

| | SEQ ID NO: 9 | | CDR |
|---|---|---|---|
| V_{H} | positions | CDR protein sequence | SEQ ID NO |
| HCDR1 | 76-99 | GGSFNFYF | 10 |
| HCDR2 | 151-171 | ITHSGSA | 11 |
| HCDR3 | 286-345 | AGLPIDYDTVHLGYYPMDV | 12 |

Antibody NDS.1: V_{L}
DIQMTQSPSSLSASVGDRVTITCRASQGIRNYLAWYQQKPGKVPKLLIYGASTLQSGVPSRFS
GSGSGTDFSLTISSLQPEDIATYFCQEYNNAPRTFGQGTKVEIK (SEQ ID NO: 13)

| | SEQ ID NO: 13 | | CDR |
|---|---|---|---|
| V_{L} | positions | CDR Sequence | SEQ ID NO |
| LCDR1 | 77-94 | QGIRNY | 14 |
| LCDR2 | 146-154 | GAS | 15 |
| LCDR3 | 263-289 | QEYNNAPRT | 16 |

Antibody NDS.3: V_{H}
EVQLVESGGGLVKPGGSLRLSCAASGFTFSDYSMTWIRQIPGKGLEWLSYTTSSGRPIFYAD
SVKGRFTMSRDNAKMSVYLQMNSLRDADSAVYYCARGDPHYIWGTYLDSWGPGTLVTVS
S (SEQ ID NO: 17)

| | SEQ ID NO: 17 | | CDR |
|---|---|---|---|
| V_{H} | positions | CDR protein sequence | SEQ ID NO |
| HCDR1 | 76-99 | GFTFSDYS | 18 |
| HCDR2 | 151-174 | TTSSGRPI | 19 |
| HCDR3 | 289-333 | ARGDPHYIWGTYLDS | 20 |

Antibody NDS.3: V_{L}
QSALTQPPSASGSPGQSVTISCTGTTSDFGDHNYVSWYQQRPGEAPKLIIYDVSKRPSGVPDR
FSGSKSGNTASLTVSRLQADDEANYYCSSIEGSNTLLFGGGTKLTVL (SEQ ID NO: 21)

| | SEQ ID NO: 21 | | CDR |
|---|---|---|---|
| V_{L} | positions | CDR Sequence | SEQ ID NO |
| LCDR1 | 76-102 | TSDFGDHNY | 22 |
| LCDR2 | 154-162 | DVS | 23 |
| LCDR3 | 271-300 | SSIEGSNTLL | 24 |

Antibody NDS.1.2: V_{H}
QVQLQQWGAGLLKPSETLSLTCDVYGGAFNFYYWTWIRQSPGRGLEWLAEITHSGSATYTP
SLKSRLTLAVDTSKGQFSLKLTSVTAADTAVYYCAALPIDYDTIYLGYYTMDVWGQGTPVA
VSS (SEQ ID NO: 25)

| | SEQ ID NO: 25 | | CDR |
|---|---|---|---|
| V_{H} | positions | CDR protein sequence | SEQ ID NO |
| HCDR1 | 76-99 | GGAFNFYY | 26 |
| HCDR2 | 151-171 | ITHSGSA | 27 |
| HCDR3 | 286-342 | AALPIDYDTIYLGYYTMDV | 28 |

Antibody NDS.1.2: V_{L}
DIQMTQSPSSLSASLGDRVTITCRASQGISSYLAWYQQRPGKVPKLLIYAASTLQSGVSPRFS
GSGSGTDFSLTISSLQPEDVAIYFCQEYDSAPRTFGQGTKVEIK (SEQ ID NO: 29)

| | SEQ ID NO: 29 | | CDR |
|---|---|---|---|
| V_{L} | positions | CDR protein sequence | SEQ ID NO |
| LCDR1 | 76-93 | QGISSY | 30 |
| LCDR2 | 145-153 | AAS | 31 |
| LCDR3 | 262-288 | QEYDSAPRT | 32 |

Antibody NDS.6: V_{H}
QVQLVQSGPEVKKPGASVKVSCKASGYNFNRYGLTWVRQAPGQGLEWMGWISTYNSDTI
YAREFRGRVTLTTDTSTRTAYMELRSLISADTAVYYCARDNVEEAFPISYYFDHWGQGTLV
SVSS (SEQ ID NO: 33)

| | SEQ ID NO: 33 | | CDR |
|---|---|---|---|
| V_{H} | positions | CDR protein sequence | SEQ ID NO |
| HCDR1 | 103-126 | GYNFNRYG | 34 |
| HCDR2 | 178-201 | ISTYNSDT | 35 |
| HCDR3 | 316-369 | ARDNVEEAFPISYYFDH | 36 |

Antibody NDS.6: V_{L}
QSALTQPRSVSGSPGQSVTISCTGTSSDVGLYNYVSWYQHHPGKAPKFLIYDVTKRPSGVPD
RFSGSKSGNTASLTISGLQAEDEADYYCCSYVGSYILVFGGGTKVTVL (SEQ ID NO: 37)

| | SEQ ID NO: 37 | | CDR |
|---|---|---|---|
| V_{L} | positions | CDR Sequence | SEQ ID NO |
| LCDR1 | 98-124 | SSDVGLYNY | 38 |
| LCDR2 | 176-184 | DVT | 39 |
| LCDR3 | 293-322 | CSYVGSYILV | 40 |

Antibody NDS.8: V_{H}
EVQLVESGGGLVKPGGSLRLSCAASGFTFSTYTMNWVRQAPGKGLEWVSSISSSGNYMYY
ADSVKGRFTISRDSAKNSLSLQMTSLSAEDTAVYYCARDKWIWDAFDIWGQGTMVTVSS
(SEQ ID NO: 41)

| | SEQ ID NO: 41 | | CDR |
|---|---|---|---|
| V_{H} | positions | CDR Sequence | SEQ ID NO |
| HCDR1 | 130-153 | GFTFSTYT | 42 |
| HCDR2 | 205-228 | ISSSGNYM | 43 |
| HCDR3 | 343-378 | ARDKWIWDAFDI | 44 |

Antibody NDS.8: V_{L}
FMTQSPFSLSVSPGERATLSCRASQSVRSKLAWYQQKPGQAPRLLISGASTRATGVPPRFSGS
GSGTEFTFTISSLQSEDFAVYYCQQYNDWPRTFGQGTRVEIK (SEQ ID NO: 45)

| | SEQ ID NO: 45 | | CDR |
|---|---|---|---|
| V_{L} | positions | CDR protein sequence | SEQ ID NO |
| LCDR1 | 74-91 | QSVRSK | 46 |
| LCDR2 | 143-151 | GAS | 47 |
| LCDR3 | 260-286 | QQYNDWPRT | 48 |

Antibody NDS.5: V_{H}
QLRLQESGPGLVKPSETLSLTCTVSGGSITSSPYYWGWIRQPPGKGLEWIGSIYYSGSTYYNP
SLRSRVTISLDTSMNQFSLKLSSVTAADTAVYYCAEEYYDFWRTQDWGQGTLVTVSS (SEQ
ID NO: 49)

| | SEQ ID NO: 49 | | CDR |
|---|---|---|---|
| V_{H} | positions | CDR Sequence | SEQ ID NO |
| HCDR1 | 185-214 | GGSITSSPYY | 50 |
| HCDR2 | 266-286 | IYYSGST | 51 |
| HCDR3 | 401-436 | AEEYYDFWRTQD | 52 |

Antibody NDS.5: V_{L}
EIVLTQSPGTLSLSPGERATISCRASQSVSGRNLAWYQQKPGQAPRLLIYAASNRATGIPDRF
SGSGSGTDFTLTISRLEPEDFAVYYCQQYGTSKTFGQGTKVEIK (SEQ ID NO: 53)

| | SEQ ID NO: 53 | | CDR |
|---|---|---|---|
| V_{H} | positions | CDR protein sequence | SEQ ID NO |
| LCDR1 | 72-92 | QSVSGRN | 54 |
| LCDR2 | 144-152 | AAS | 55 |
| LCDR3 | 261-284 | QQYGTSKT | 56 |

Antibody NDS.7: V_{H}
QVQLVQSGVEVKKPGASVKVSCKASGYTFTRHGITWVRQAPGQGLEWMGWIAPYNGDTI
YAEKFQARVTLTMDTSTNTVYLEMRSLSSDDTAVYYCGRDSVEEAFPISYYFDYWGQGTL
VTVSS (SEQ ID NO: 57)

| | SEQ ID NO: 57 | | CDR |
|---|---|---|---|
| V_{H} | positions | CDR Sequence | SEQ ID NO |
| HCDR1 | 103-126 | GYTFTRHG | 58 |
| HCDR2 | 178-201 | IAPYNGDT | 59 |
| HCDR3 | 316-366 | GRDSVEEAFPISYYFDY | 60 |

Antibody NDS.7: V_{L}
QSVLTQPRSVSGSPGQSVTISCTGTTNEIGIYNFVSWYQHHPGEAPRLIMYDVSKRPSGVPDR
FSGSKSGNTASLTISGLQTEDEAAYYCCSYVGSYKLLFGGGTKLTVL (SEQ ID NO: 61)

| | SEQ ID NO: 61 | | CDR |
|---|---|---|---|
| V_{L} | positions | CDR protein sequence | SEQ ID NO |
| LCDR1 | 97-123 | TNEIGIYNF | 62 |
| LCDR2 | 175-183 | DVS | 63 |
| LCDR3 | 292-321 | CSYVGSYKLL | 64 |

a. Monoclonal Antibody NDS.1.1

In some aspects, the antibody or antigen binding fragment is based on or derived from the NDS.1.1 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.1.1 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 5, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, 7, and 8, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, 7, and 8 respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 5, such as 95%, 96%, 97%, 98% or 99% identical to SEQ ID NO: 5, and the antibody or antigen binding fragment and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 1 and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 5, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 1 and 5, respectively, and specifically binds to the underside of NA, and inhibits an influenza virus, such as an H3N2 influenza virus.

In some aspects, the disclosed antibodies inhibit viral entry and/or replication.

b. Monoclonal Antibody NDS.1

In some aspects, the antibody or antigen binding fragment is based on or derived from the NDS.1 antibody, and specifically binds to the underside of NA, and inhibits an influenza virus, such as an H3N2 influenza virus. In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.1 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 9, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 9 and 13, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 10, 11, and 12, respectively, and/or a V_{L} comprising a LCDR1, a LCDR2, and a LCDR3 as set forth as SEQ ID NOs: 14, 15 and 16, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 10, 11, and 12, respectively, a V_{L} comprising a LCDR1, a LCDR2, and a LCDR3 as set forth as SEQ ID NOs: 14, 15 and 16, respectively, wherein the V_{H} comprises an amino acid sequence at least 90% identical to SEQ ID NO: 9, such as 95%, 96%, 97%, 98% or 99% identical to SEQ ID NO: 9, and wherein the V_{L} comprises an amino acid sequence at least 90% identical to SEQ ID NO: 13, such as 95%, 96%, 97%, 98% or 99% identical to SEQ ID NO: 13, and the antibody or antigen binding fragment specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 9, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 13, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 9 and 13, respectively, and specifically binds to the underside of NA, and inhibits an influenza virus, such as an H3N2 influenza virus.

In some aspects, the disclosed antibodies inhibit viral entry and/or replication.

c. Monoclonal Antibody NDS.3

In some aspects, the antibody or antigen binding fragment is based on or derived from NDS.3 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.3 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 17, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 21, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 17 and 21, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 18, 19, and 20, respectively, and/or a V_{L} Comprising a LCDR1, a LCDR2, and a LCDR3 as set forth as SEQ ID NOs: 22, 23, and 24, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 18, 19, and 20, respectively, a V_{L} comprising a LCDR1, a LCDR2, and a LCDR3 as set forth as SEQ ID NOs: 22, 23, and 24, respectively, wherein the V_{H} comprises an amino acid sequence at least 90% identical to SEQ ID NO: 17, such as 95%, 96%, 97%, 98% or 99% identical to SEQ ID NO: 17, and wherein the V_{L} comprises an amino acid sequence at least 90% identical to SEQ ID NO: 21, such as 95%, 96%, 97%, 98% or 99% identical to SEQ ID NO: 21, and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 17, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 21, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 17 and 21, respectively, and specifically binds to the underside of NA, and inhibits an influenza virus, such as an H3N2 influenza virus.

In some aspects, the disclosed antibodies inhibit viral entry and/or replication.

d. Monoclonal Antibody NDS.1.2

In some aspects, the antibody or antigen binding fragment is based on or derived from the NDS.1.2 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.1.2 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 25, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 29, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 25 and 29, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 26, 27, and 28, respectively, and/or a V_{L} comprising a LCDR1, a LCDR2, and a LCDR3 as set forth as SEQ ID NOs: 30, 31, and 32, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 26, 27, and 28, respectively, a V_{L} comprising a LCDR1, a LCDR2, and a LCDR3 as set forth as SEQ ID NOs: 30, 31, and 32, respectively, wherein the V_{H} comprises an amino acid sequence at least 90% identical to SEQ ID NO: 25, such as 95%, 96%, 97%, 98% or 99% identical to SEQ ID NO: 25, and wherein the V_{L} comprises an amino acid sequence at least 90% identical to SEQ ID NO: 29, such as 95%, 96%, 97%, 98% or 99% identical to SEQ ID NO: 29, and the antibody or antigen binding fragment and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 25, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 29, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 25 and 29, respectively, and specifically binds to the underside of NA, and inhibits an influenza virus, such as an H3N2 influenza virus.

In some aspects, the disclosed antibodies inhibit viral entry and/or replication.

e. Monoclonal Antibody NDS.6

In some aspects, the antibody or antigen binding fragment is based on or derived from the NDS.6 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.6 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 33, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 37, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 33 and 37, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 34, 35, and 36, respectively, and/or a V_{L} comprising a LCDR1, a LCDR2, and a LCDR3 as set forth as SEQ ID NOs: 38, 39, and 40, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 34, 35, and 36, respectively, a V_{L} comprising a LCDR1, a LCDR2, and a LCDR3 as set forth as SEQ ID NOs: 38, 39, and 40, respectively, wherein the V_{H} comprises an amino acid sequence at least 90% identical to SEQ ID NO: 33, such as 95%, 96%, 97%, 98% or 99% identical to SEQ ID NO: 33, and wherein the V_{L} comprises an amino acid sequence at least 90% identical to SEQ ID NO: 37, such as 95%, 96%, 97%, 98% or 99% identical to SEQ ID NO: 37, and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 33, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 37, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 33 and 37, respectively, and specifically binds to the underside of NA, and inhibits an influenza virus, such as an H3N2 influenza virus.

In some aspects, the disclosed antibodies inhibit viral entry and/or replication.

f. Monoclonal Antibody NDS.8

In some aspects, the antibody or antigen binding fragment is based on or derived from the NDS.8 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.8 antibody, and specifically binds to the underside of NA, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 41, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 45, and specifically binds to the underside of NA, and inhibits an influenza virus, such as an H3N2 influenza virus. Tn additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 41 and 45, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 42, 43, and 44, respectively, and/or a V_{L} comprising a LCDR1, a LCDR2, and a LCDR3 as set forth as SEQ ID NOs: 46, 47, and 48, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 42, 43, and 44, respectively, a V_{L} comprising a LCDR1, a LCDR2, and a LCDR3 as set forth as SEQ ID NOs: 46, 47, and 48, respectively, wherein the V_{H} comprises an amino acid sequence at least 90% identical to SEQ ID NO: 41, such as 95%, 96%, 97%, 98% or 99% identical to SEQ ID NO: 41, and wherein the V_{L} comprises an amino acid sequence at least 90% identical to SEQ ID NO: 45, such as 95%, 96%, 97%, 98% or 99% identical to SEQ ID NO: 45, and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 41, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 45, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 41 and 45, respectively, and specifically binds to the underside of NA, and inhibits an influenza virus, such as an H3N2 influenza virus.

In some aspects, the disclosed antibodies inhibit viral entry and/or replication.

g. Monoclonal Antibody NDS.5

In some aspects, the antibody or antigen binding fragment is based on or derived from the NDS.5 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.5 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 49, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 53, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 49 and 53, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 50, 51, and 52, respectively, and/or a V_{L} comprising a LCDR1, a LCDR2, and a LCDR3 as set forth as SEQ ID NOs: 54, 55, and 56, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 50, 51, and 52, respectively, a V_{L} comprising a LCDR1, a LCDR2, and a LCDR3 as set forth as SEQ ID NOs: 54, 55, and 56, respectively, wherein the V_{H} comprises an amino acid sequence at least 90% identical to SEQ ID NO: 49, such as 95%, 96%, 97%, 98% or 99% identical to SEQ ID NO: 49, and wherein the V_{L} comprises an amino acid sequence at least 90% identical to SEQ ID NO: 53, such as 95%, 96%, 97%, 98% or 99% identical to SEQ ID NO: 53, and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 49, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 53, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 49 and 53, respectively, and specifically binds to the underside of NA, and inhibits an influenza virus, such as an H3N2 influenza virus.

In some aspects, the disclosed antibodies inhibit viral entry and/or replication.

h. Monoclonal Antibody NDS.7

In some aspects, the antibody or antigen binding fragment is based on or derived from the NDS.7 antibody, and specifically binds to the underside of NA, and inhibits an influenza virus, such as an H3N2 influenza virus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the NDS.7 antibody, and specifically binds to the underside of NA, and inhibits an influenza virus, such as an H3N2 influenza virus.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 57, and specifically binds to the underside of NA, and inhibits an influenza virus, such as an H3N2 influenza virus. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 61, and specifically binds to the underside of NA, and inhibits an influenza virus, such as an H3N2 influenza virus. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 57 and 61, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 58, 59, and 60, respectively, and/or a V_{L} comprising a LCDR1, a LCDR2, and a LCDR3 as set forth as SEQ ID NOs: 62, 63, and 64, respectively, and specifically binds to the underside of NA, and inhibits an influenza virus, such as an H3N2 influenza virus.

In some aspects, the antibody or antigen binding fragment comprises a V_{H} comprising a HCDR1, a HCDR2, and a HCDR3 as set forth as SEQ ID NOs: 58, 59, and 60, respectively, a V_{L} comprising a LCDR1, a LCDR2, and a LCDR3 as set forth as SEQ ID NOs: 62, 63, and 64, respectively, wherein the V_{H} comprises an amino acid sequence at least 90% identical to SEQ ID NO: 57, such as 95%, 96%, 97%, 98% or 99% identical to SEQ ID NO: 57, and wherein the V_{L} comprises an amino acid sequence at least 90% identical to SEQ ID NO: 61, such as 95%, 96%, 97%, 98% or 99% identical to SEQ ID NO: 61, and specifically binds to the underside of NA, and inhibits an influenza virus, such as an H3N2 influenza virus. In this aspect, variations due to sequence identify fall outside the CDRs.

In some aspects, the antibody or antigen binding fragment comprises a V_{H} comprising the amino acid sequence set forth as SEQ ID NO: 57, and specifically binds to the underside of NA, and inhibits an influenza virus, such as an H3N2 influenza virus. In more aspects, the antibody or antigen binding fragment comprises a V_{L} comprising the amino acid sequence set forth as SEQ ID NO: 61, and specifically binds to the underside of NA, and inhibits an influenza virus, such as an H3N2 influenza virus. In some aspects, the antibody or antigen binding fragment comprises a V_{H} and a V_{L} comprising the amino acid sequences set forth as SEQ ID NOs: 57 and 61, respectively, and specifically binds to the underside of NA, and inhibits an influenza virus, such as an H3N2 influenza virus.

In some aspects, the disclosed antibodies inhibit viral entry and/or replication.

i. Additional Antibodies

In some examples, antibodies that bind to an epitope of interest can be identified based on their ability to cross-compete (for example, to competitively inhibit the binding of, in a statistically significant manner) with the antibodies provided herein in binding assays. In other examples, antibodies that bind to an epitope of interest can be identified based on their ability to cross-compete (for example, to competitively inhibit the binding of, in a statistically significant manner) with the one or more of the antibodies provided herein in binding assays.

Human antibodies that bind to the same epitope on the underside of NA, to which the disclosed antibodies bind can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Additional human antibodies that bind to the same epitope can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope can also be isolated by screening combinatorial libraries for antibodies with the desired binding characteristics. For example, by generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Competitive binding assays, similar to those disclosed in the examples section below, can be used to select antibodies with the desired binding characteristics.

### 2. Additional Description of Antibodies and Antigen Binding Fragments

An antibody or antigen binding fragment of the antibodies disclosed herein can be a human antibody or fragment thereof. Chimeric antibodies are also provided. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgA, IgM or an IgG antibody, such as IgG_{G}, IgG₂, IgG₃, or IgG₄. The class of an antibody that specifically binds to the underside of NA can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} or C_{H} chain. For example, an antibody that specifically binds the spike protein, that was originally IgG may be class switched to an IgA. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to the underside of NA is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### (a) Binding Affinity

In several aspects, the antibody or antigen binding fragment specifically binds the underside of NA with an affinity (e.g., measured as K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 230° C.). In a non-adsorbent plate (NUNC™ Catalog #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT™-20; PerkinElmer) is added, and the plates are counted on a TOPCOUNT™ gamma counter (PerkinElmer) for ten minutes. Concentrations of each Fab that give less than or equal to 20% of maximal binding are chosen for use in competitive binding assays.

In another assay, K_{D} can be measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k^{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Affinity can also be measured by high throughput SPR using the Carterra LSA.

### (b) Multispecific Antibodies

In some aspects, a multi-specific antibody, or a bi-specific antibody, such as a dual variable domain antibody (DVD-IG™) is provided that comprises an antibody or antigen binding fragment that specifically binds the underside of NA, as provided herein. In other aspects, any suitable method can be used to design and produce a bispecific antibody, such as crosslinking two or more antibodies, antigen binding fragments (such as scFvs) of the same type or of different types. Exemplary methods of making multispecific antibodies, such as bispecific antibodies, include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). In some aspects, the multispecific antibody, such as the bispecific antibody, includes a disclosed antibody that specifically binds the underside of NA, or an antigen binding fragment thereof, and an antibody or antigen binding fragment thereof that specifically binds HA of an H1, H2, H3, H4, H5, H6, H7, H8, H9, H10, H11, H12, H13, H14, H15, H16, H17 or H18 subtype influenza virus. In particular examples, the antibody or antigen binding fragment thereof binds HA of a H2 or H3 subtype influenza virus, In some aspects, the antibody or antigen binding fragment thereof specifically binds an HA stem domain, an HA lateral patch domain, or HA vestigial esterase domain.

The multi-specific antibody may have any suitable format that allows for binding to the underside of NA by the antibody or antigen binding fragment as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and Brühl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

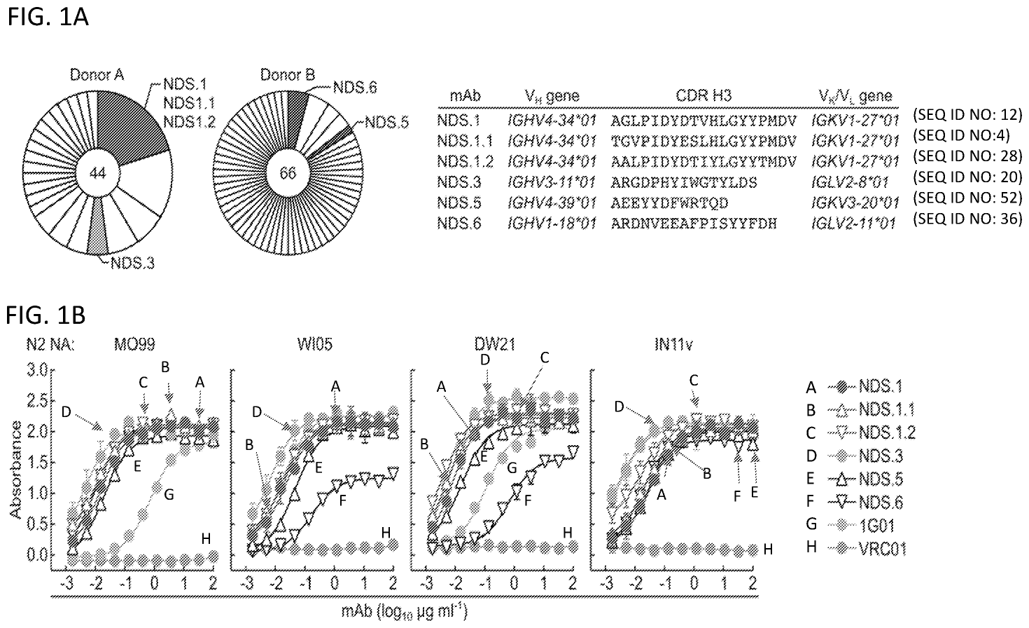

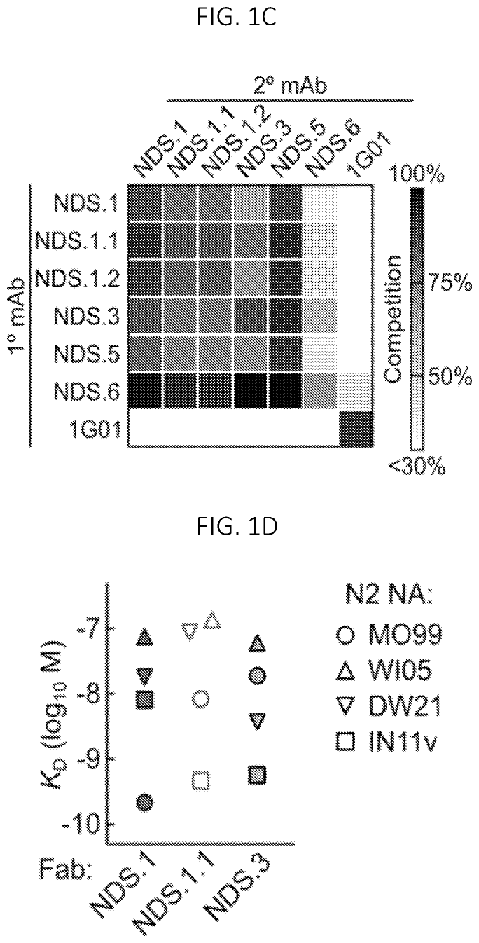

The bispecific tetravalent immunoglobulin known as the dual variable domain immunoglobulin or DVD-immunoglobulin molecule is disclosed in Wu et al., MAbs. 2009; 1:339-47, doi: 10.4161/mabs.1.4.8755, incorporated herein by reference. See also Nat Biotechnol. 2007 November; 25(11):1290-7. doi: 10.1038/nbt1345. Epub 2007 Oct. 14, also incorporated herein by reference. A DVD-immunoglobulin molecule includes two heavy chains and two light chains. Unlike IgG, however, both heavy and light chains of a DVD-immunoglobulin molecule contain an additional variable domain (VD) connected via a linker sequence at the N-termini of the VH and VL of an existing monoclonal antibody (mAb). Thus, when the heavy and the light chains combine, the resulting DVD-immunoglobulin molecule contains four antigen recognition sites, see Jakob et al., Mabs 5: 358-363, 2013, incorporated herein by reference, see FIG. 1 for schematic and space-filling diagrams. A DVD-immunoglobulin molecule functions to bind two different antigens on each DFab simultaneously.

The outermost or N-terminal variable domain is termed VD1 and the innermost variable domain is termed VD2; the VD2 is proximal to the C-terminal CH1 or CL. As disclosed in Jakob et al., supra, DVD-immunoglobulin molecules can be manufactured and purified to homogeneity in large quantities, have pharmacological properties similar to those of a conventional IgG₁, and show in vivo efficacy. Any of the disclosed monoclonal antibodies can be included in a DVD-immunoglobulin format.

### (c) Antigen Binding Fragments

Antigen binding fragments are encompassed by the present disclosure, such as Fab. F(ab′)₂, and Fv which include a heavy chain and V_{L} and specifically bind the underside of NA. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### (d) Variants

In some aspects, amino acid sequence variants of the antibodies and bispecific antibodies provided herein are provided. For example, it may be desirable to improve the binding affinity and/or other biological properties of the antibody or bispecific antibody. Amino acid sequence variants of an antibody may be prepared by introducing appropriate modifications into the nucleotide sequence encoding the antibody V_{H} domain and/or V_{L} domain, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some aspects, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield. In some aspects, the amino acid substitutions are not in the CDRs of the antibody.

In some aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In more aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In further aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 21.

In yet other aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 25. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 29.

In more aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 33. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 37.

In more aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 41. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 45.

In more aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 49. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 53.

In more aspects, the heavy chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 57. In some aspects, the light chain of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 61.

In some aspects, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody/bispecific antibody, or the light chain of the antibody/bispecific antibody, or the heavy and light chains of the antibody/bispecific antibody, compared to known framework regions, or compared to the framework regions of the antibody, and maintain the specific binding activity for the epitope of the underside of NA. In some aspects, the amino acid substitutions re not in the CDRs of the antibody.

In some aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some aspects of the variant V_{H} and V_{L} sequences provided above, each CDR either is unaltered, or contains no more than one, two or three amino acid substitutions. In some aspects of the variant V_{H} and V_{L} sequences provided above, only the framework residues are modified so the CDRs are unchanged.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for the spike protein. In particular examples, the V_{H} amino acid sequence is one of SEQ ID NOs:1, 9, 17, 25, 33, 41, 49, or 57, respectively. In other examples, the V_{L} amino acid sequence is one of SEQ ID NOs: 5, 13, 21, 29, 37, 45, 53, or 61, respectively.

In some aspects, an antibody disclosed herein, an antigen binding fragment, or bispecific antibody is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered.

Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody may be made in order to create antibody variants with certain improved properties.

In one aspect, variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function.

See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fe region are also provided. Such antibody variants may have improved CDC function. Such antibody variants are described, e.g., in WO 1997/30087; WO 1998/58964; and WO 1999/22764.

In several aspects, the constant region of the antibody or bispecific antibody comprises one or more amino acid substitutions to optimize in vivo half-life of the antibody. The serum half-life of IgG Abs is regulated by the neonatal Fc receptor (FcRn). Thus, in several aspects, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies and antigen binding fragments can be linked to or comprise an Fc polypeptide including any of the substitutions listed above, for example, the Fc polypeptide can include the M428L and N434S substitutions.

In some aspects, an antibody or bispecific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies, antigen binding fragments, and bispecific antibodies that specifically bind to the underside of NA, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ²¹¹⁵, ³²P, ¹⁴C, ³H and ³⁵S and other labels, target moieties, enzymes and ligands, etc. The choice of a particular effector molecule or detectable marker depends on the particular target molecule or cell, and the desired biological effect.

The procedure for attaching a detectable marker to an antibody, antigen binding fragment, or bispecific antibody, varies according to the chemical structure of the effector. Polypeptides typically contain a variety of functional groups, such as carboxyl (—COOH), free amine (−NH₂) or sulfhydryl (—SH) groups, which are available for reaction with a suitable functional group on a polypeptide to result in the binding of the effector molecule or detectable marker. Alternatively, the antibody, antigen binding fragment, or bispecific antibody, is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody, antigen binding fragment, or bispecific antibody, and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or bispecific antibody can be determined.

The antibody, antigen binding fragment or bispecific antibody can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody, antigen binding fragment, or bispecific antibody, can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody, antigen binding fragment, or bispecific antibody, may also be conjugated with biotin, and detected through indirect measurement of avidin or streptavidin binding. It should be noted that the avidin itself can be conjugated with an enzyme or a fluorescent label.

The antibody, antigen binding fragment or bispecific antibody, can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody, antigen binding fragment, or bispecific antibody, may also be labeled with a predetermined polypeptide epitope recognized by a secondary reporter (such as leucine zipper pair sequences, binding sites for secondary antibodies, metal binding domains, epitope tags).

The antibody, antigen binding fragment or bispecific antibody, can also be conjugated with a radiolabeled amino acid, for example, for diagnostic purposes. For instance, the radiolabel may be used to detect an influenza virus by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody, antigen binding fragment, or bispecific antibody in a conjugate can range, for example, from 1 to 20 moieties per antibody or antigen binding fragment. In some aspects, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules) encoding the amino acid sequences of antibodies, antigen binding fragments, bispecific antibodies, and conjugates that specifically bind to the underside of NA, as disclosed herein, are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several aspects, nucleic acid molecules can encode the V_{H}, the V_{L}, or both the V_{H} and V_{L} (for example in a bicistronic expression vector) of a disclosed antibody or antigen binding fragment. In some aspects, the nucleic acid molecules encode an scFv. In several aspects, the nucleic acid molecules can be expressed in a host cell (such as a mammalian cell) to produce a disclosed antibody or antigen binding fragment. Nucleic acid molecules encoding an scFv are provided.

The genetic code can be used to construct a variety of functionally equivalent nucleic acid sequences, such as nucleic acids which differ in their sequence but which encode the same antibody sequence, or encode a conjugate or fusion protein including the V_{L} and/or V_{H} nucleic acid sequence.

In a non-limiting example, an isolated nucleic acid molecule encodes the V_{H} of a disclosed antibody. In another non-limiting example, the nucleic acid molecule encodes the V_{L} of a disclosed antibody. In further non-limiting examples, the nucleic acid molecule can encode a bi-specific antibody, such as in DVD-immunoglobulin format.

Nucleic acid molecules encoding the antibodies, antigen binding fragments, bispecific antibodies, and conjugates that specifically bind to the underside of NA can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L}, (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A. 85(16):5879-5883, 1988; McCafferty et al., Nature, 348:552-554, 1990; Kontermann and Dübel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that hind specifically to the underside of NA and another antigen. The encoded V_{H} and V_{L} optionally can include a furin cleavage site between the V_{H} and V_{L} domains. Linkers can also be encoded, such as when the nucleic acid molecule encodes a bi-specific antibody in DVD-IG™ format.

One or more DNA sequences encoding the antibodies, antigen binding fragments, bispecific antibodies, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and mycloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies, antigen binding fragments, and bispecific antibodies (such as DVD-immunoglobulin antibodies) described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, bispecific antibodies, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, bispecific antibodies, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

i. Therapeutic Methods

Methods are disclosed herein for the prevention or treatment of an influenza infection. Prevention and treatment can include inhibition of infection with N2 subtype influenza viruses, such as, but not limited to circulating H3N2 viruses, related viruses, any naturally emerging or engineered influenza viruses that contain the underside of NA of an N2 subtype influenza virus, and the HA of a different subtype, including H2, or H3 subtypes. In some aspects, the disclosed method can be used to treat an H3N2, H2N2, H1N2, and/or H9N2 infection in a subject. The methods can include administering to a subject a therapeutically effective amount of a monoclonal antibody, an antigen binding fragment thereof, or a bispecific antibody, as disclosed above, or a nucleic acid encoding the antibody or antigen binding fragment. In some aspects, the methods include contacting a cell with an effective amount of a monoclonal antibody, or antigen binding fragment thereof, that specifically binds the underside of NA of an N2 subtype influenza virus. In one example the subject is a human or other mammal. In one example, the subject is a bird, such as a chicken or turkey.

In some aspects, the methods include selecting a subject has an infection with an N2 subtype influenza virus for treatment. The method can include selecting a subject has an infection with an H3N2, H2N2, H1N1 and/or H9N2 subtype influenza virus for treatment. In one specific non-limiting example, a subject is selected that has an H3N2 infection. In another specific non-limiting example, a subject is selected that has an H2N2 infection. In a further specific non-limiting example, a subject is selected that has an H1N2 infection. In yet another specific non-limiting example, a subject is selected that has an H9N2 infection. All of these subjects can be treated using the disclosed methods.

The disclosed antibodies, antigen binding fragments and nucleic acids can be used as emergency prophylaxis to protect individuals in the proximity of a developing influenza outbreak, or when encountering an increased risk of exposure. Thus, the methods can include selecting a subject at risk of exposure to influenza, such as a subject that is at particular risk, such as the elderly, a pediatric subject, an immunocompromised individual, a pregnant woman, a person with heart disease or a health care worker.

In other aspects, methods are disclosed for ameliorating one or more symptoms associated with an influenza virus infection. In more aspects, the method includes administering an antibody, an antigen-binding fragment thereof, or a bispecific antibody that specifically binds the underside of NA. The method can also include administering to the subject a nucleic acid molecule encoding the antibody, antigen binding fragment, or bispecific antibody. The method can include selecting a subject with an influenza virus infection, such as an N2 subtype influenza virus infection.

For treatment, the influenza virus infection does not need to be completely eliminated for the composition to be effective. The symptoms of the viral infection can be reduced, such as fever, cough, upper respiratory symptoms, and/or lower respiratory symptoms. In some aspects, there is a shortened duration of symptoms. Thus, in some aspects, the duration of symptoms is reduced by at least 10%, at least 20%, at least 50%, at least 60%, at least 70%, at least 80%, at least 90%, at least 95%, at least 98%, or 100%, as compared to the duration of symptoms in the absence of the composition. In some aspects, a composition can decrease the infection in a population by a desired amount, for example by at least 10%, at least 20%, at least 50%, at least 60%, at least 70%, at least 80%, at least 90%, at least 95%, at least 98%, or 100%, as compared to the rate of infection in the absence of the composition. In addition, a composition in some examples decreases viral titer by at least 10%, at least 20%, at least 50%, at least 60%, at least 70%, at least 80%, at least 90%, at least 95%, at least 98%, or 100% in a subject.

In one aspect, administration of the antibody, antigen binding fragment, bispecific antibody (or nucleic acid encoding the antibody, antigen binding fragment, or bispecific antibody) results in a reduction in the establishment of a virus infection and/or reducing subsequent disease progression in a subject. A reduction in the establishment of an infection, and/or a reduction in subsequent disease progression can encompass a statistically significant reduction in viral activity and/or replication. In some aspects, methods are disclosed for treating a subject with an existing influenza virus infection. In other aspects, methods are disclosed for preventing an influenza virus infection. These methods include administering to the subject a therapeutically effective amount of an antibody, antigen binding fragment thereof, or specific antibody, or a nucleic acid encoding the antibody, antigen binding fragment, or bispecific antibody, thereby preventing or treating the viral infection. The method can prevent, delay or reduce symptoms of an influenza virus infection, such as fever, vomiting, body ache, sore throat, coughing, nasal congestion, headache, and fatigue. The influenza virus can be an H3N2 influenza virus.

A therapeutically effective amount of an antibody that specifically binds the underside of NA, antigen binding fragment, or bispecific antibody (or the nucleic acid encoding the antibody, antigen binding fragment or bispecific antibody) will depend upon the severity of the disease and/or infection and the general state of the patient's health. A therapeutically effective amount of the antibody can provide either subjective relief of a symptom(s) or an objectively identifiable improvement as noted by the clinician or other qualified observer. These compositions can be administered in conjunction with another therapeutic agent, either simultaneously or sequentially. For any application, the antibody, antigen binding fragment, or nucleic acid encoding the antibody or antigen binding fragment can be combined with additional anti-viral therapy.

In some aspects, the subject is also administered an effective amount of an additional agent, such as anti-viral agent. The methods can include administration of one on more additional agents known in the art. The method can include administering a neuraminidase inhibitor (such as oseltamivir, or zanamivir). The method can include administering to the subject a therapeutically effective amount of an M2 inhibitor, such as amantadine and/or rimantadine. The method can include administering to the subject a therapeutically effective amount of peramivir. The subject can be hydrated and administered balancing electrolytes. The subject can be administered intravenous inmuunoglobulins.

For prevention or treatment, the subject can also be administered a therapeutically effective amount of one or more additional antibodies or antigen binding fragments that specifically bind an additional influenza protein. The influenza protein can be an H1N1, H1N2, H5N1, H6N1, H7N2, H7N3, H7N7, H7N9, H9N2, H10N7 or H10N8 influenza A virus protein or can be an influenza B virus protein. In specific aspects, the influenza protein can be an H1N1, H3N2, H2N2, H5N1 or H7N9 influenza virus protein. The influenza virus protein can be a NA or a HA. The influenza virus protein can be NA of an N1, N2, N3, N4, N5, N6, N7, N8, N9, N10 or N11 subtype influenza virus, or the HA of any influenza A subtype, H1-H18.

The subject can be administered a therapeutically effective amount of more than one antibody, such as 1, 2, 3, 4, 5, 6, 7, 8, 9, or 10 antibodies with specificity for different influenza virus proteins. The subject can be administered a therapeutically effective amount of one or more antibodies that bind NA of a different (not N2) subtype influenza virus. Combinations of the antibodies disclosed herein are also of use.

Thus, in some examples, the subject can be administered a therapeutically effective amount of one or more additional antibodies or antigen binding fragments that specifically binds either NA or HA of a specific influenza A virus, such as H1N1, H1N2, H2N2, H3N2, H5N1, H6N1, H7N2, H7N3, H7N7, H7N9, H9N2, H10N7 or H10N8. In other non-limiting examples, the subject can also be administered a therapeutically effective amount of an antibody or antigen binding fragment that specifically binds HA (or NA) of H1N1 or a therapeutically effective amount of an antibody or antigen binding fragment that specifically binds HA (or NA) of H5N1, or a therapeutically effective amount of an antibody or antigen binding fragment that specifically binds HA (or NA) of H7N9 influenza virus.

These aspects and examples are not mutually exclusive. Thus, the subject can be administered 1, 2, 3, 4, 5, 6, 7, 8, 9 or 10 additional antibodies or antigen binding fragments that specifically bind an influenza virus protein. The antibodies can be administered in one or more pharmaceutical compositions. The antibodies can be administered in a single pharmaceutical composition. Thus, pharmaceutical compositions are provided herein that include the disclosed antibodies and/or antigen binding fragments and additional antibodies that specifically bind an influenza virus protein.

Single or multiple administrations of the compositions including the antibody, antigen binding fragment, bispecific antibody, or nucleic acid encoding the antibody, antigen binding fragment, or bispecific antibody that are disclosed herein, are administered depending on the dosage and frequency as required and tolerated by the patient. In any event, the composition should provide a sufficient quantity to effectively treat the patient. The dosage can be administered once but may be applied periodically until either a therapeutic result is achieved or until side effects warrant discontinuation of therapy.

In one example, a dose of the antibody is infused for thirty minutes every other day. In this example, about one to about ten doses can be administered, such as three or six doses can be administered every other day. In a further example, a continuous infusion is administered for about five to about ten days. The subject can be treated at regular intervals, such as daily, until a desired therapeutic result is achieved. Generally, the dose is sufficient to treat or ameliorate symptoms or signs of disease without producing unacceptable toxicity to the patient.

Compositions are provided that include one or more of the antibody or antigen binding fragment that specifically bind the underside of NA and nucleic acids encoding these antibodies (and antigen binding fragments and bispecific antibodies) that are disclosed herein in a carrier. As disclosed above, the compositions can include antibodies or antigen binding fragments that specifically bind an influenza virus protein.

Compositions are provided that include one or more disclosed antibody, antigen binding fragment, bispecific antibody, conjugate, or nucleic acid molecule encoding such molecules, that are disclosed herein in a pharmaceutically acceptable carrier. In some aspects, the composition comprises two, three, four or more antibodies, antigen binding fragments, or bispecific antibodies, that specifically bind the underside of NA. The compositions are useful, for example, for example, for the inhibition or detection of an influenza virus infection, such as an H3N2 infection. In some aspects, the composition includes the NDS.1.1, NDS.1, NDS.3, NDS.1.2, NDS.6, NDS.8, NDS.5, or NDS.7 antibody disclosed herein, or an antigen binding fragment thereof, or a bispecific antibody thereof. In some aspects, the composition comprises two, three, four or more antibodies that specifically bind the underside of NA. The compositions are useful, for example, for example, for the inhibition or detection of an influenza virus infection, such as an H3N2 infection.

The compositions can be prepared in unit dosage forms, such as in a kit, for administration to a subject. The amount and timing of administration are at the discretion of the administering physician to achieve the desired purposes. The antibody, antigen binding fragment, bispecific antibody, conjugate, or nucleic acid molecule encoding such molecules can be formulated for systemic or local administration. In one example, the, antigen binding fragment, bispecific antibody, conjugate, or nucleic acid molecule encoding such molecules, is formulated for parenteral administration, such as intravenous administration.

In some aspects, the antibody, antigen binding fragment, bispecific antibody, or conjugate thereof, in the composition is at least 70% (such as at least 75%, at least 80%, at least 85%, at least 90%, at least 95%, at least 96%, at least 97%, at least 98% or at least 99%) pure. In some aspects, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the antibody, antigen binding fragment, bispecific antibody, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

The compositions can be prepared in unit dosage forms for administration to a subject. The amount and timing of administration are at the discretion of the treating physician to achieve the desired purposes. The antibody, antigen binding fragment, bispecific antibody, and/or nucleic acid can be formulated for systemic or local administration. In one example, the antibody and/or nucleic acid is formulated for parenteral administration, such as intravenous administration. In some aspects, administration is intramuscular. Compositions also can be formulated for intranasal administration as a liquid or an aerosol. In some aspects, the antibody can be administered to the respiratory tract by using a nebulizer.

Active ingredients can also be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Specifically, liposomes containing the immunogens or antibodies can be prepared by such methods as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556. The reverse-phase evaporation method can be used with a lipid composition comprising phosphatidylcholine, cholesterol and PEG-derivatized phosphatidylethanolamine (PEG-PE). Liposomes are extruded through filters of defined pore size to yield liposomes with the desired diameter. Polypeptides such as antibodies or antibody fragments can be conjugated to the liposomes as described, for example, in Martin et al., J. Biol. Chem., 257:286-288 (1982) via a disulfide interchange reaction.

The compositions for administration can include a solution of the antibody that specifically binds the underside of NA, or an antigen binding fragment thereof, or a bispecific antibody, dissolved in a pharmaceutically acceptable carrier, such as an aqueous carrier. A variety of aqueous carriers can be used, for example, buffered saline and the like. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibodies, antigen binding fragments, or bispecific antibodies in these formulations can vary widely, and will be selected primarily based on fluid volumes, viscosities, body weight and the like in accordance with the particular mode of administration selected and the subject's needs. In some aspects, administration is intravenous.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker. Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems. Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

A typical pharmaceutical composition for intravenous administration includes about 0.1 to 10 mg/kg of antibody (or antigen binding fragment) per day, or 0.5 to 15 mg/kg of antibody per day. Dosages from 0.1 up to about 100 mg/kg per subject per day may be used, particularly if the agent is administered to a secluded site and not into the circulatory or lymph system, such as into a body cavity or into a lumen of an organ. Exemplary doses include 1 to 10 mg/kg, such as 2 to 8 mg/kg, such as 3 to 6 mg/kg. Actual methods for preparing administrable compositions will be known or apparent to those skilled in the art and are described in more detail in such publications as Remington's Pharmaceutical Science, 19th ed., Mack Publishing Company, Easton, PA (1995).

Antibodies, antigen binding fragments, and bispecific antibodies may be provided in lyophilized form and rehydrated with sterile water before administration, although they are also provided in sterile solutions of known concentration. The antibody (antigen binding fragment, or bispecific antibody) solution is then added to an infusion bag containing 0.9% sodium chloride, USP, and typically administered at a dosage of from 0.1 to 50 mg/kg or 0.5 to 15 mg/kg of body weight. Exemplary doses include 1 to 10 mg/kg, such as 2 to 8 mg/kg, such as 3 to 6 mg/kg.

Antibodies, antigen binding fragments thereof, and bispecific antibodies can be administered by intravenous infusion. Doses of the antibody, antigen binding fragment, or bispecific antibody vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some aspects, the dose of the antibody, antigen binding fragment or bispecific antibody can be from about 0.5 mg/kg to about 5 mg/kg, such as a dose of about 1 mg/kg, about 2 mg/kg, about 3 mg/kg, about 4 mg/kg or about 5 mg/kg. The antibody, antigen binding fragment, or bispecific antibody is administered according to a dosing schedule determined by a medical practitioner. In some examples, the antibody, antigen binding fragment or bispecific antibody is administered weekly, every two weeks, every three weeks or every four weeks.

A therapeutically effective amount of a nucleic acid encoding the antibody, an antigen binding fragment thereof, or bispecific antibody can be administered to a subject in need thereof. One approach to administration of nucleic acids is direct immunization with plasmid DNA, such as with a mammalian expression plasmid. The nucleotide sequence encoding the antibody or fragment thereof can be placed under the control of a promoter to increase expression of the molecule. Immunization by nucleic acid constructs is well known in the art and taught, for example, in U.S. Pat. Nos. 5,643,578, and 5,593,972 and 5,817,637. U.S. Pat. No. 5,880,103 describes several methods of delivery of nucleic acids to an organism. The methods include liposomal delivery of the nucleic acids.

In another approach to using nucleic acids, an antibody, antigen binding fragment thereof, or bispecific antibody can also be expressed by attenuated viral hosts or vectors or bacterial vectors, which can be administered to a subject. Recombinant vaccinia virus, adeno-associated virus (AAV), herpes virus, retrovirus, cytomegalovirus, poxvirus or other viral vectors can be used to express the antibody. For example, vaccinia vectors are described in U.S. Pat. No. 4,722,848. BCG (Bacillus Calmette Guerin) provides another vector for expression of the disclosed antibodies (see Stover, Nature 351:456-460, 1991).

In one aspect, a nucleic acid encoding the antibody, an antigen binding fragment, or bispecific antibody is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 mg/kg to about 50 mg/kg, and typically are about 0.005 mg/kg to about 5 mg/kg (see, e.g., U.S. Pat. No. 5,589,466).

In some aspects, a subject is administered DNA or RNA encoding a disclosed antibody, antigen binding fragment, or bispecific antibody, to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, antigen binding fragments thereof, or bispecific antibody can be placed under the control of a promoter to increase expression. The methods include liposomal delivery of the nucleic acids. Such methods can be applied to the production of an antibody, or antigen binding fragments thereof. In some aspects, a disclosed antibody or antigen binding fragment is expressed in a subject using the pVRC8400 vector (described in Barouch et al., J. Virol., 79(14), 8828-8834, 2005, which is incorporated by reference herein).

In several aspects, a subject (such as a human subject at risk of a influenza virus infection or having an influenza virus infection) can be administered an effective amount of an AAV viral vector that comprises one or more nucleic acid molecules encoding a disclosed antibody, antigen binding fragment, or bispecific antibody. The AAV viral vector is designed for expression of the nucleic acid molecules encoding a disclosed antibody, antigen binding fragment, or bispecific antibody, and administration of the effective amount of the AAV viral vector to the subject leads to expression of an effective amount of the antibody, antigen binding fragment, or bispecific antibody in the subject. Non-limiting examples of AAV viral vectors that can be used to express a disclosed antibody, antigen binding fragment, or bispecific antibody in a subject include those provided in Johnson et al., Nat. Med., 15(8):901-906, 2009 and Gardner et al., Nature, 519(7541):87-91, 2015, each of which is incorporated by reference herein in its entirety.

In one aspect, a nucleic acid encoding a disclosed antibody, antigen binding fragment, or bispecific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed antibody, antigen binding fragment, or bispecific antibody, conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit an influenza virus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

In aspects, the antibody, antigen binding fragment, bispecific antibody or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

A typical composition for intravenous administration comprises about 0.01 to about 30 mg/kg of antibody, antigen binding fragment, bispecific antibody, or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some aspects, the composition can be a liquid formulation including one or more antibodies, antigen binding fragments, or bispecific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

ii. Detection Methods and Vaccine Identification

Methods are provided herein for the detection of the expression of NA, in vitro or in vivo. In one example, expression of NA is detected in a sample, and can be used to detect an infection with an influenza virus, such as, but not limited to, H3N2. Biological samples include sections of tissues, for example, frozen sections taken for histological purposes, tissue from biopsies, autopsies and pathology specimens. Biological samples also include body fluids, such as blood, serum, plasma, sputum, tears, saliva, spinal fluid, nasopharyngeal secretions or urine, or stool. The sample can be a nasal wash, a throat swab, nasal swab, or a lung aspirate. In other aspects, the sample is an environmental sample.

In aspects, methods are provided for detecting the presence of an influenza virus, such as an N2 subtype influenza virus. The disclosed methods can be used to detect the presence of an H3N2, H2N2, H1N2 and/or an H9N2 influenza virus, such as in a sample, for example an environmental or biological sample.

The presence of the influenza virus is detected in a sample suspected of containing the virus, wherein the method includes contacting the sample with an antibody disclosed herein, or an antigen binding fragment thereof, and determining binding of the antibody or antigen binding fragment to the virus in the sample. In these methods, binding of the antibody or antigen binding fragment to virus in the sample is indicative of the presence of the virus in the sample. In one aspect, the sample is a biological sample. The binding of the antibody or antigen binding fragment can be quantitated.

There are three genera of influenza virus (A, B, and C), all of which infect humans. Influenza A viruses are classified based on the viral surface proteins NA and hemagglutinin (HA). Eighteen HA subtypes (or serotypes) and eleven NA subtypes of influenza A virus have been identified. Specific influenza strain isolates can be identified by a standard nomenclature specifying virus type, geographical location where first isolated, sequential number of isolation, year of isolation, and HA and NA subtype. The serotypes that are known to have been associated with deaths are H1N1, H1N2, H2N2, H3N2, H5N1, H5N6, H7N7, H7N9, H9N2, and H10N8. The presently disclosed antibodies and antigen binding fragments specifically bind N2, and can be used to identify an N2 subtype influenza virus. In some aspects, the antibody specifically binds N2, and can be used to identify an H1N2, H2N2, H3N2, or H9N2 infection. In some aspects, the disclosed methods include determining the H subtype of the influenza virus.

In some aspects, methods are disclosed herein for identifying an influenza virus as an H3N2 influenza virus, and/or distinguishing an N2 subtype virus from other subtypes of influenza viruses, such as other influenza A viruses, such as an N1 subtype influenza virus, N3 subtype influenza virus, N4 subtype influenza virus, N5 subtype influenza virus, N6 subtype influenza virus, N7 subtype influenza virus, N8 subtype influenza virus, N9 subtype influenza virus, N10 subtype influenza virus and/or an N11 subtype influenza virus. In a specific non-limiting example, the methods identify the virus as an N2 subtype influenza virus and/or distinguish the N2 subtype influenza virus from other N subtypes. Thus, in some aspects, the method distinguishes a particular influenza from other influenza viruses, such that it identifies and influenza virus as being an N2 subtype influenza virus.

The method includes contacting the sample with an antibody disclosed herein that specifically binds NA of an N2 subtype influenza virus, and determining binding of the antibody to the virus in the sample. In some aspects, binding of the antibody to virus in the sample is indicative of the presence of an N2 subtype influenza virus in the sample. In one aspect, the sample is a biological sample. In other aspects, the sample is an environmental sample.

The methods can also include contacting the sample with an antibody or antigen binding fragment thereof that specifically binds HA of an H1, H2, H3, H4, H5, H6, H7, H8, H9, H10, H11, H12, H13, H14, H15, H16, H17 or H18 subtype influenza virus, and determining binding of the antibody to the virus in the sample. In some aspects, binding of the antibody to virus in the sample is indicative of the presence of a particular subtype of influenza virus. In specific non-limiting examples, the monoclonal antibody or antigen binding fragment specifically binds H3.

In some aspects, methods are disclosed herein for identifying an influenza virus as an H3N2 virus, and/or distinguishing an H3N2 virus from other influenza viruses, such as other influenza A viruses, such as an H1N1, an H1N2 virus, an H2N2 virus, an H5N1 virus, an H6N1 virus, an H7N2 virus, an H7N3, an H7N7 virus, an H7N9 virus, an H9N2 virus, an H10N7 virus, and/or an H10N8 virus. Thus, in some aspects, the method distinguishes a particular influenza from other influenza virus, such that it identifies and influenza virus as being an H3N2 influenza virus.

In several aspects, a method is provided for detecting an N2 subtype influenza virus infection in a subject. The disclosure provides a method for detecting an N2 subtype influenza virus in a biological sample, wherein the method includes contacting a biological sample with the antibody or antigen binding fragment that specifically binds the underside of NA of an N2 subtype influenza virus, specifically the antibodies and antigen binding fragments disclosed herein, under conditions conducive to the formation of an immune complex, and detecting the immune complex, to detect the presence of a NA polypeptide in the biological sample. In additional aspects, detection of the NA polypeptide in the sample detects or confirms a diagnosis of an N2 subtype influenza virus infection. In some aspects, an antibody fragment, such as an Fv fragment, a Fab, or any of the antigen binding fragments disclosed above, can be utilized in the disclosed methods. The amount of N2 subtype influenza virus can be quantitated by methods known in the art.

The detection of NA of an N2 subtype influenza virus can be achieved, for example, by contacting a sample to be tested, optionally along with a control sample, with the antibody (or antigen binding fragment) under conditions that allow for formation of a complex between the antibody or antigen binding fragment and the virus. Complex formation is then detected (e.g., using an ELISA). The detection of the immune complex indicates that an N2 subtype influenza virus is present in the sample, and/or that the subject has an N2 subtype influenza virus infection. When using a control sample along with the test sample, complex is detected in both samples and any statistically significant difference in the formation of complexes between the samples is indicative of the presence of the N2 subtype influenza virus in the test sample. In some aspects, the control is a standard value. In other aspects, the control is the binding of the monoclonal antibody or antigen binding fragment to a control sample comprising a known quantity of neuraminidase. The control can also be the binding of the monoclonal antibody or antigen binding fragment to a control sample from a subject known not to have an influenza virus infection.

The methods can also include contacting the sample with an antibody or antigen binding fragment thereof that specifically binds HA of an H1, H2, H3, H4, H5, H6, H7, H8, H9, H10, H11, H12, H13, H14, H15, H16, H17 or H18 subtype influenza virus, and determining binding of the antibody to the virus in the sample. In some aspects, binding of the antibody to virus in the sample is indicative of the presence of a particular subtype of influenza virus. In specific non-limiting examples, the monoclonal antibody or antigen binding fragment specifically binds H1, H2, H3 or H5. The method includes contacting a biological sample with the antibody or antigen binding fragment that specifically binds HA under conditions conducive to the formation of an immune complex, and detecting the immune complex, to detect the presence of a HA polypeptide in the biological sample. In some aspects, the antibody or antigen binding fragment is labeled. In specific non-limiting examples, the monoclonal antibody specifically binds HA of an H3 subtype influenza virus. Thus, the method can detect the presence of an H3N2 influenza virus and/or distinguish the presence of the H3N2 influenza virus from other influenza viruses. Thus, the method can detect the presence of an H3N2 influenza virus and/or distinguish the presence of the H3N2 influenza virus from other influenza viruses.

In some aspects, the disclosed antibodies can be used as standards in enzyme linked lectin assays (ELLA). An ELLA measures the ability of antibodies or pharmaceutical agents to inhibit NA activity. In the assay, a NA substrate, fetuin, is immobilized to the surface of wells of a protein-binding plate. NA is added to the substrate in the presence of different concentrations of antibody and incubated for a time period that allows the enzyme to cleave sialic acid from fetuin in control wells that do not contain antibody or NA inhibitors. Peanut agglutinin (PNA) conjugated to a label, such as, but not limited to, horse radish peroxidase is then added to the wells. The binding of PNA to molecules in the well that become exposed by the removal of sialic acid indicates how much neuraminidase activity is present. If antibodies inhibit this activity, the signal is in proportion to the amount of antibody present. In one example, the assay is used to measure the potency of NA-specific antibodies, derivatives of antibodies or other NA inhibitors, with the disclosed antibodies included in the ELLA as a reference standard.

In some aspects, the disclosed antibodies are used to test vaccines. For example to test if a vaccine composition can induce NA inhibiting antibodies, such as, but not limited to, inhibition of NA of H3N2 influenza viruses. Thus provided herein is a method for testing a vaccine, wherein the method includes contacting a sample containing the vaccine, such as an NA polypeptide, or a recombinant attenuated virus, with the antibody (or antigen binding fragment) under conditions conducive to the formation of an immune complex, and detecting the immune complex. The detection of the immune complex confirm the vaccine will be effective. In one example, the detection of the immune complex in the sample indicates that the vaccine component, such as such as an NA antigen assumes a conformation capable of inducing neutralizing antibodies.

In some aspects, an antibody (or antigen binding fragment) is directly labeled with a detectable label. In another aspect, the antibody (or antigen binding fragment) that binds NA (the first antibody) is unlabeled and a second antibody or other molecule that can bind the antibody that binds NA is utilized. As is well known to one of skill in the art, a second antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody are described above, and include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H. The antibodies can be directly labeled. In some aspects, a second antibody is used that that specifically binds a disclosed isolated monoclonal antibody or antigen binding fragment to form a immune complex, and detecting the binding of the second antibody.

The immunoassays and method disclosed herein can be used for a number of purposes. Kits for detecting NA of an N2 subtype influenza virus will typically comprise an antibody (or antigen binding fragment) that specifically binds the underside of NA of an N2 subtype influenza virus, for example, any of the antibodies or antigen binding fragments disclosed herein. In some aspects, an antibody fragment, such as an Fv fragment or a Fab is included in the kit. In a further aspect, the antibody is labeled (for example, with a fluorescent, radioactive, or an enzymatic label).

In one aspect, a kit includes instructional materials disclosing means of use. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one aspect, the diagnostic kit comprises an immunoassay. Although the details of the immunoassays may vary with the particular format employed, the method of detecting the NA of an N2 subtype influenza virus in a biological sample generally includes the steps of contacting the biological sample with an antibody or antigen binding fragment which specifically binds, under immunologically reactive conditions, to underside of NA of the N2 subtype influenza virus. The antibody is allowed to specifically bind under immunologically reactive conditions to form an immune complex, and the presence of the immune complex (bound antibody) is detected directly or indirectly. Methods for detection and quantification of NA are disclosed above.

The following examples are provided to illustrate certain particular features and/or aspects. These examples should not be construed to limit the disclosure to the particular features or aspects described.

### EXAMPLES

Circulating seasonal influenza viruses cause substantial morbidity and mortality and pose global public health and economic challenges (Iuliano et al., Lancet 391: 1285-1300 (2018); Putri et al., Vaccine 36: 3960-3966 (2018)). In addition, a new influenza pandemic on the scale last seen in 1918 could potentially claim tens or hundreds of millions of lives worldwide (Spreeuwenbert et al., Am. J. Epidemiol. 187: 2561-2567 (2018)). Current influenza vaccines mainly induce antibodies against the surface glycoprotein hemagglutinin (HA) that can block viral attachment to its host receptors and/or viral membrane fusion to the host cell (Chen et al., Cell 173: 417-429 (2018)). These antibodies primarily target the immunodominant head region of HA, which undergoes antigenic drift in response to immune pressure (Air et al., Contrib. Microbiol. Immunol. 8: 20-59 (1987)). As a result, vaccine-elicited antibodies provide protection against vaccine-matched strains of the virus, but they confer little cross-protection to other strains or subtypes. Considerable effort in influenza vaccine design has been directed at developing immunogens that elicit broadly neutralizing antibody responses to subdominant epitopes of HA, like the HA stem domain, and discovery of new broadly conserved viral sites of vulnerability remains a priority for next generation influenza vaccine development (Wei et al., Nat. Rev. Drug Discov. 19: 239-252 (2020); McMillan et al., Vaccines (Basel) 9 (2021); Kanekiyo et al., Cold Spring Harb. Prospect. Med a038448 (2020)).

Neuraminidase (NA) is a less abundant but essential surface protein of influenza viruses (Krammer et al., MBio 9: e02332-17 (2018)). NA cleaves sialic acid from glycoproteins and glycolipids on the host cell surface, allowing progeny viruses to be released from infected cells (McAuley et al., Front. Microbiol. 10: 39 (2019)). In addition, NA enables virions to navigate through the respiratory tract by cleaving off decoy receptors present in the mucus and mucosal membranes (Matrosovich et al., J. Virol. 78: 12665-12667 (2019)). The catalytic activity of NA is one the main targets of influenza antivirals, and NA inhibitors have been widely utilized to treat influenza in humans for more than two decades.

Recently, two human broadly cross-reactive anti-NA monoclonal antibodies (mAbs), 1G01 and DA03E17, were described (Stadlbauer et al., Science 366: 499-504 (2019); Yasuhara et al., Nat. Commun. 13: 6602 (2022)). 1G01 was isolated from an H3N2-infected individual shortly after the onset of symptoms. This antibody targets the catalytic site of NA with an extended complementary-determining region H3 loop (CDR H3) and inhibits virtually all NA subtypes of influenza A and B viruses in vivo and in vitro. DA03E17, isolated using the hybridoma method from an individual who was infected with A/H1N1pdm09 virus, binds to or near the enzymatic active site and shows similarly broad protection in vivo and in vitro. Another group discovered two influenza B anti-NA mAbs that broadly protect against both Yamagata- and Victoria-lineage viruses by targeting the catalytic site of NA (Madsen et al., Immunity 53: 852-863 (2020)). Furthermore, the murine mAb CD6 protects against pdmH1N1 by binding to an epitope on the “side” of the NA globular head domain that spans two neighboring protomers and inhibiting the catalytic activity of NA through steric hindrance (Wan et al., Nat. Commun. 6: 6114 (2015)). Similar mAbs have been isolated from an individual upon natural H7N9 influenza infection (Gilchuk et al., Cell Host Microbe 26: 715-728 (2019); Zhu et al., Cell Host Microbe 26: 729-738 (2019)) and a mouse infected with H1N8 virus (Saito et al., J. Virol. 68: 1790-1796 (1994)).

As the murine mAb CD6 was induced by infection and its almost perpendicular angle of approach to the NA head, it is plausible that the NA head may be flexible on the viral surface. Recently, Casalino et al. described the dynamics of influenza glycoproteins in a crowded protein environment using mesoscale all-atom molecular dynamics simulations and revealed that the NA globular head domain can tilt up to 90 degrees on the virion, exposing the lateral as well as the underside surface of NA globular head to immune recognition (Casalino et al., ACS Cent. Sci. 8:1646-1663 (2022)). High-resolution cryo-electron tomography (cryo-ET) has shown similar tilting by the SARS-CoV-2 spike protein on the surface of virions (Ke et al., Nature 588: 498-502 (2020)) and by influenza HA on a detergent micelle (Benton et al., Proc. Natl. Acad. Sci 115: 10112-10117 (2018)). Furthermore, structural studies of viral glycoproteins with similar three-dimensional folds as influenza NA, notably Nipah G, have revealed dissociation of the four head domains and antibody binding to the “underside” of the head (Wang et al., Science 375: 1373-1378 (2022)). Thus, different epitopes on NA may be targeted by biologically relevant antibodies.

The isolation of human NA-specific mAbs is disclosed herein. Several of these antibodies target conserved epitopes on the underside of the globular head of N2 NAs. These mAbs inhibit H3N2 and H2N2 virus propagation in vitro and confer protection against H3N2 infection in mice in both prophylactic and therapeutic settings. Cryo-EM structures of two such mAbs in complex with N2 NA defined in atomic detail the epitopes on the NA “dark side” (NDS). Characterization of such antibodies and their epitopes can aid the design of NA-based antigens for next-generation influenza vaccines (Burton et al., Nat. Rev. Immunol. 2: 706-713 (2002); Rappuoli et al. J. Exp. Med. 213: 469-481 (2016)), and these antibodies can be used therapeutically and in biologic assays.

### Example 1: Materials and Methods

Single-cell sorting, immunoglobulin amplification, sequencing and mAb production: Cryopreserved peripheral blood mononuclear cell (PBMC) samples were thawed and stained with monoclonal antibodies against human CD3 (BioLegend), CD14 (BioLegend), CD56 (BioLegend), CD19 (BioLegend or Beckam Coulter), CD21 (BD), CD27 (BioLegend), CD38 (BioLegend), IgM (BD) and IgG (BD). The NA probes of N2 Å/Wisconsin/67/2005 (WI05) or N2 Å/Darwin/09/2021 (DW21) were conjugated with either AF488 or AF647 fluorochrome, respectively, according to the manufacturer's instruction (Microscale Protein Labeling Kit, Thermo Fisher Scientific) prior to use in flow cytometry. Non-B cells and dead cells were gated out with CD3, CD14, CD56 and Aqua (cell viability dye) staining. Memory B cells were gated on as CD19+, CD38^{low}, IgM⁻ and IgG⁺ or IgG⁺ and CD20⁺. NA-binding memory B cells were single-cell sorted into 96-well plates (Bio-Rad) using an FACS Aria Instrument (BD). Plates were subjected to reverse transcription PCR and cDNA were used for PCR to amplify immunoglobulin (Ig) heavy and light chain genes as described previously (see, for example, Doria-Rose et al., J. Virol. 91(1): 76-91, Oct. 14, 2015). PCR products were sequenced by Sanger sequencing, and sequences were analyzed with the IMGT V-Quest (imgt.org/IMGT_vquest). Paired Ig heavy and light chain sequences were synthesized and subcloned into IgG1 heavy chain and kappa or lambda light chain backbone expression vectors, respectively (GenScript). Antibodies were expressed recombinantly by transient transfection of Ig expression plasmids in Expi293 cells (ThermoFisher Scientific) with EXPIFECTAMINE™ 293 Transfection Kit (ThermoFisher Scientific). Supernatant was harvested 5 days post transfection and antibody was purified by using Protein A Sepharose (Cytiva).

Human specimens: Human PBMC samples used in this study were obtained under the study, VRC 200, a protocol for apheresis and specimen collection procedures to obtain plasma, PBMCs and other specimens for research studies (ClinicalTrials.gov identifier NCT00067054) at the National Institutes of Health (NIH) Clinical Center by the Vaccine Research Center (VRC) Clinical Trials Program, National Institute of Allergy and Infectious Disease (NIAID), NIH in Bethesda, MD. The trial protocol was reviewed and approved by the NIAID Institutional Review Board. Informed consent was obtained from every enrolled participant and conduct of the study complied with all relevant ethical regulations. Compensation was provided to participants for their time and effort related to participation in this clinical trial research study.

Cell lines: Expi293 cells, purchased from Thermo Fisher Scientific (Catalog A14527), were cultured in Expi293 Expression Medium (Life Technologies) at 37° C. with 8% CO₂ and agitating at 120 rpm. MDCK-SIAT1-PB1 cells used for virus propagation, growth inhibition assay, and NA-star assay were described previously Creanga et al., Nat. Commun. 12: 1722 (2021)). MDCK-SIAT1-PB1 cells were maintained in DMEM supplemented with 10% FBS, geneticin (1 mg ml⁻¹) and puromycin (0.25 μg ml⁻¹) at 37° C. with 5% CO₂.

Viruses: Rescue and propagation of replication-restricted reporter (R3) influenza viruses used in this study was described previously (Creanga et al., Nat. Commun. 12: 1722 (2021)). Briefly, H3N2 viruses (i.e., A/Aichi/2/1968, A/Philippines/2/1982. A/Moscow/10/1999, A/Wisconsin/67/2005, A/Indiana/10/2011, A/Switzerland/9715293/2013, A/Darwin/6/2021) used in this study are R3 viruses in which PB1 segment was modified to encode a fluorescent reporter, whereas A/Singapore/1/1957 H2N2 virus is rewired R3 in which HA coding region is inserted between PB1 segment genome packaging signals and the fluorescent reporter is inserted between HA segment genome packaging signals.

Protein expression and purification: Recombinant NA proteins were expressed and purified as described previously. Briefly, NA constructs were expressed by transient transfection in Expi293 cells (ThermoFisher Scientific) using the EXPIFECTAMINE™293 Transfection Kit (ThermoFisher Scientific). Cell culture supernatants were harvested 5 days post-transfection, cleared, and filtered. Proteins were purified from clarified supernatants by immobilized metal affinity chromatography (IMAC) using Ni-Sepharose High-Performance resin (Cytiva). After incubating for 2 hours at room temperature, bound proteins were eluted using an elution buffer (50 mM Tris-HCl, 0.5 M NaCl, 300 mM imidazole, pH 8). Proteins were further purified by size exclusion chromatography (SEC) into phosphate-buffer saline (PBS) using a Superdex 200 Increase 10/300 column (Cytiva).

Enzyme-linked immunosorbent assay (ELISA): MAXISORP™ ELISA plates (Nunc) were coated with 2 μg ml⁻¹ of recombinant NA protein and incubated overnight at 4° C. Plates were washed with PBS and 0.1% Tween 20 (PBS-T) and blocked with 5% skim milk in PBS at 37° C. for 1 hour. MAbs were diluted to 0.1 mg ml⁻¹ and serially diluted threefold and added to the plates for 30 minutes at 37° C. After washing, HRP-conjugated secondary antibody (anti-human; Southern Biotech) was added and incubated for another 30 minutes at 37° C. Plates were developed with KPL TMB substrate and the reaction was stopped by the addition of 0.5 M H₂SO₄. Absorbance was measured at 450 nm (Biotek Neo2 plate reader).

Biolayer Interferometry (BLI): BLI experiments were performed by using the Octet HTX instrument (Sartorius). HIS1K biosensors (Sartorius) were hydrated in PBS prior to use. Recombinant N2 NA proteins derived from A/Moscow/10/1999, A/Wisconsin/67/2005, A/Indiana/08/2011 and A/Darwin/09/2021 were immobilized on hydrated HIS1K biosensors through their hexahistidine tag at their N-termini. After brief (60 seconds) equilibration in assay buffer (25 mM Tris, 150 mM NaCl, 1% BSA, pH 8) the biosensors were dipped into a two-fold dilution series of NDS.1, NDS.1.1 or NDS.3 Fabs for 300 seconds. Starting concentration of Fabs was 400-800 nM. Biosensors were then dipped in the assay buffer to allow Fabs to dissociate from NA for 600 seconds. All assay steps were performed at 30° C. with agitation set at 1,000 rpm. Baseline correction was carried out by subtracting the measurements recorded for a sensor loaded with the corresponding NA in the same buffer with no Fab. Data analysis and curve fitting were done with the Octet analysis software (version 12). Experimental data were fitted with the binding equations describing a 1:1 (Langmuir model) interaction.

Influenza replication inhibition neuraminidase-based assay (IRINA): The viruses were diluted in Opti-MEM (Gibco) and 45 μl of titrated virus was added to 45 μl of Opti-MEM into a 96-well plate and incubated for 1 h at 37° C. MCDK-STAT1-PB1 cells were trypsinized, washed once with PBS and resuspended in Opti-MEM. Thirty μl of MDCK-SIAT1-PB1 cells at a concentration of 1×10⁶ cells ml-were added to each well containing the diluted virus. Twenty-five μl of virus-cell mixture was added to each 384 well in quadruplicate and incubated for 20-24 h at 37° C. with 5% CO₂. Next day, mAbs were diluted in the assay buffer (NA-Fluor Influenza Neuraminidase Assay Kit; Thermo Fisher Scientific) at a starting concentration of 50 μg ml⁻¹. Culture supernatant of the 384-well plate was discarded and replenished with 25 μl diluted mAbs in quadruplicate. The plates were incubated for 1 h at 37° C. with 5% CO₂, and 25 μL of assay substrate (NA-Fluor Influenza Neuraminidase Assay Kit; Thermo Fisher Scientific) was added to each well and incubated for another 1 h at 37° C. with 5% CO₂. Reaction was then stopped by adding 50 μl of stop solution (NA-Fluor Influenza Neuraminidase Assay Kit; Thermo Fisher Scientific), and the plates were read using an excitation wavelength range of 350 nm to 365 nm and an emission wavelength range of 440 nm to 460 nm. Control wells without the virus infection were used for background subtraction.

Enzyme-linked lectin assay (ELLA): MaxiSorp ELISA plates were coated with fetuin at 2.5 μg well⁻¹ in the coating buffer (KPL coating solution; SeraCare), sealed and stored at 4° C. overnight or until further use. Next day, the plates were washed with PBS-T. A two-fold serial dilution of virus was prepared in a separate 96-well plate with a starting dilution of 1:10 in sample diluent (PBS, 1% BSA, 0.5% Tween-20). Fifty μl of each virus dilution was added to the washed plate, sealed and incubated for 2 h at 37° C. The plates were washed six times with PBS-T, and 100 μl well⁻¹ of HRP-conjugated peanut agglutinin (Sigma) at a concentration of 1 μg ml⁻¹ was added to each plate. The plates were incubated for 2 h at room temperature and washed three times with PBS-T. The plates were developed with 100 μl well⁻¹ SigmaFast OPD in the dark at room temperature and the reaction was stopped after 10 min with 100 μl well⁻¹ of 0.5 M H₂SO₄. The plates were read at a wavelength of 490 nm (Biotek Neo2 plate reader).

For the neuraminidase inhibition assay, the plates were coated and blocked as described above. In a separate plate mAbs were diluted to a starting concentration of 50 μg ml⁻¹, serially two-fold diluted in sample diluent. Fifty μl well⁻¹ of diluted virus was added to the fetuin-coated plate, and 50 μl well⁻¹ of diluted mAb was added on top. Fifty μl well⁻¹ of sample diluent was added on diluted virus for positive control wells, whereas 100 μL of sample diluent (without virus solution) was added for negative control wells. Plates were sealed, gently mixed by tapping sides of plates, and incubated for 2 h at 37° C. The plates were washed, developed, and read as described above.

Virus Inhibition assay: MAbs were serially diluted three-fold at a starting concentration of 50 μg ml⁻¹ in Opti-MEM, and the viruses were diluted in Opti-MEM supplemented with TPCK-treated trypsin (2 μg ml⁻¹). Forty-five μl of diluted mAb was incubated with 45 μl of diluted virus for 1 h at 37° C. with 5% CO₂ in 96-well plates. MCDK-SIAT1-PB1 cells were trypsinized, washed once with PBS and resuspended in Opti-MEM. Thirty μl of MDCK-SIAT1-PB1 cells at a concentration of 1×10⁶ cells ml⁻¹ were added to each well containing mAb-virus mixture. Twenty-five μl of each cell-mAb-virus mixture was added in quadruplicate to 384-well plates, and the plates were incubated for 4-5 h at 37° C. with 5% CO₂. After incubation, supernatant was removed and replenished with 25 μl of diluted mAb and 25 μl of Opti-MEM supplemented with TPCK-treated trypsin. Plates were incubated for 28-48 h at 37° C. with 5% CO₂ and fluorescent cells (i.e., reporter virus-infected cells) were detected with Celigo instrument (PerkinElmer). Results were analyzed and IC₅₀ values were calculated with GraphPad Prism. Wells with zanamivir and without mAb were used as positive (100%) and negative (0%) controls for virus inhibition, respectively.

Passive transfer: For prophylactic treatment setting, six- to eight-week old female BALB/cJ mice (n=9-10) were given 200 μl of mAbs solution at a concentration of 10, 3, or 1 mg kg⁻¹ intraperitoneally 24 h prior to challenge. Mice were anesthetized and challenged intranasally with 10×LD₅₀ of H3N2 A/Philippines/2/1982 virus. For post-exposure treatment setting, mice (n=9-10) were challenged with 5×LD₅₀ of H3N2 A/Philippines/2/1982 virus and treated 48 h later with 10 mg/kg of mAbs intraperitoneally. Body weight loss and clinical symptoms were monitored daily for 14 days post-infection and the humane endpoint was defined as a loss of 20% of the initial day 0 body weight.

Cryo-EM structure determination: Neuraminidase was mixed with the Fab fragments at a molar ratio of 1.5 Fab to 1 NA monomer. Quantifoil R 2/2 gold grids were glow-discharged using a PELCO easiGlow glow-discharger (air pressure: 0.39 mBar, current: 20 mA, duration: 30 seconds) immediately before use. Vitrification was performed at a total protein concentration of 0.15 mg/ml using a Thermo Scientific Vitrobot Mark IV plunger with the following parameters: chamber temperature of 4° C., chamber humidity of 95% and drop volume of 2.7 μl.

Datasets were collected on an FEI Titan Krios G1 electron microscope equipped with a Gatan K2 Summit direct electron detector operated in the counting mode. A single dataset, collected without tilting the stage, was used to determine the structure of the NDS.3-DW21 NA complex, which showed uniform particle distribution in the vitrified ice. In contrast, the NDS.1-IN11v NA complex demonstrated a strong preferred orientation, with only top views present in the initial dataset. To improve the angular distribution of the particles, Fab 1G01, which binds to the opposite side of the NA tetramer, was added to the NDS.1-IN11v NA complex before freezing, and datasets were collected and 0° and 30° of stage tilt. An additional dataset was recorded with the ternary complex prepared using Quantifoil R 2/2 gold grids covered with a monolayer of graphene oxide; these grids were produced in-house by following a published protocol (Han et al., J. Immunol. Methods 329: 112-124 (2008)). The total protein concentration was reduced to 0.03 mg ml⁻¹ for vitrification using graphene oxide-covered grids. All the above datasets were combined to obtain the structure of the NDS.1-1G01-IN11v NA complex.

The RELION 3.1 pipeline was used for single particle analysis (Han et al., Proc. Nat. Acad. Sci. 117: 1009-1014 (2020)), with patch-based movie frame alignment performed with MotionCor2 (Zheng et al., Nat. Methods 14: 331-332 (20178)) and contrast transfer function (CTF) parameters determined using ctffind4 (Rohou et al., J. of Struc. Biol. 192: 216-221 (2015)). Templates for automatic particle selection were obtained by 2D classification of particles picked from a small subset of micrographs using the template-free Laplacian-Gaussian filter-based approach. In the case of the NDS.1-1G01-IN11v NA complex, particles picked using the template-based approach in RELION were combined with particles selected using neural network-based picking with crYOLO (Wagner et al., Commun. Biol. 192: 216-221 (2015)), followed by elimination of duplicates. Subsequent steps included rounds of 2D classification, initial 3D volume generation using the stochastic gradient descent algorithm, 3D classification, 3D auto-refinement, particle polishing and CTF refinement. In the case of the NDS.1-1G01-IN11v NA complex, the occupancy of NDS.1 Fab in the consensus map was incomplete. Therefore, we performed symmetry expansion followed by signal subtraction to isolate a single NA monomer with bound Fabs. This was followed by 3D classification without particle re-alignment to select complexes with occupied NDS.1 Fab. The resulting subset of particles was then subjected to 3D refinement with limited angular search. Resolutions were calculated using the gold-standard approach (Henderson et al., Structure 20: 205-214 (2012)) at the Fourier shell correlation (FSC) curve threshold of 0.143. ResMap 1.1.4 was used to evaluate local resolution (Kucukelbir et al., Nat. Methods 11: 63-65 (2014)). 3D map post-processing was performed using the standard automated approach implemented in RELION as well as with DeepEMhancer (Sanchez-Garcia et al., Commun. Biol. 4: 874 (2021)).

To obtain the atomic models of the complexes, the SWISS-MODEL server (Waterhouse et al., Nucleic Acids Res. 46: W296-W303 (2018)) was used to generate homology models of NA and Fab molecules, which were then docked into the cryo-EM density using UCSF Chimera (Pettersen et al., J. Comput. Chem 25: 1605-1612 (2004)). The atomic models were refined by alternating rounds of real-space refinement in Phenix (Liebschner et al., Acta Crystallogr. D. Struct. Biol. 75: 861-877 (2019)) and model building in Coot (Emsley et al., Acta Crystallogr. D. Biol. 60: 2126-2132 (2004)). Molprobity was used for structure validation (Emsley et al., Acta Crystallogr. D. Biol. 60: 2126-2132 (2004); Davis et al., Nucleic Acids Res. 32: W615-619 (2004)). Cryo-EM map-molecular model FSC curves were obtained with phenix.mtriage (Afonine et al., Acta Crystallogr. D Struc. Biol. 74: 814-840 (2018)) at a threshold level Of 0.5. NA-Fab interfaces were determined with PDBe PISA (Krissinel et al., J. Mol. Biol. 372: 774-797 (2007)). Clustal Omega was used for protein sequence alignment (Sievers et al., Mol. Syst. Biol. 7: 539 (2011)).

Statistical analysis: All in vitro experimental data shown except for B cell sorting and cryo-EM structure determination were representative of at least two independent experiments. Conclusions from the repeated experiments that are not shown in the manuscript are the same as those from the shown experiments. The sample size in in vivo experiments was determined based on the expected heterogeneity of the samples, the significance threshold (chosen at 0.05), the expected or observed difference, as well as previous publications and our pilot studies. The chosen sample size in each experiment is sufficient to generate statistically significant results. In all experiments, unless otherwise indicated, data are shown as mean with all data points or group mean±SD, and statistical analyses were performed using GraphPad Prism (v9). Mantel-Cox test was used to compare Kaplan-Meier curves with Bonferroni correction applied. P values less than or equal to 0.05 (0.0033 after Bonferroni correction) were considered significant.

### Example 2: Isolation of Pan-N2 NA-Targeting Antibodies from Two Human Convalescent Donors

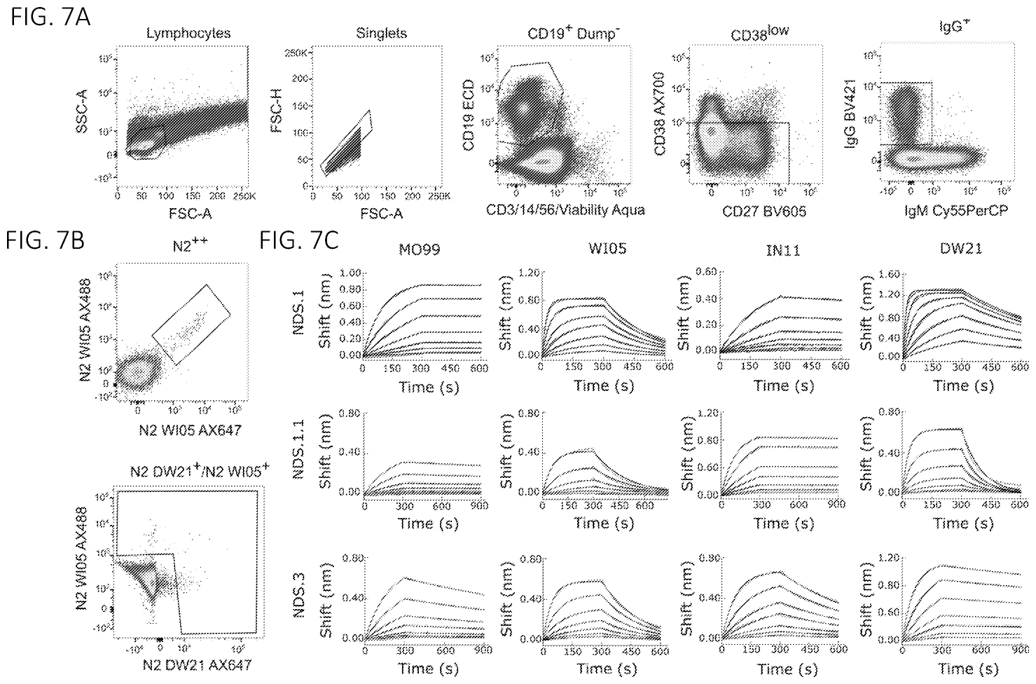

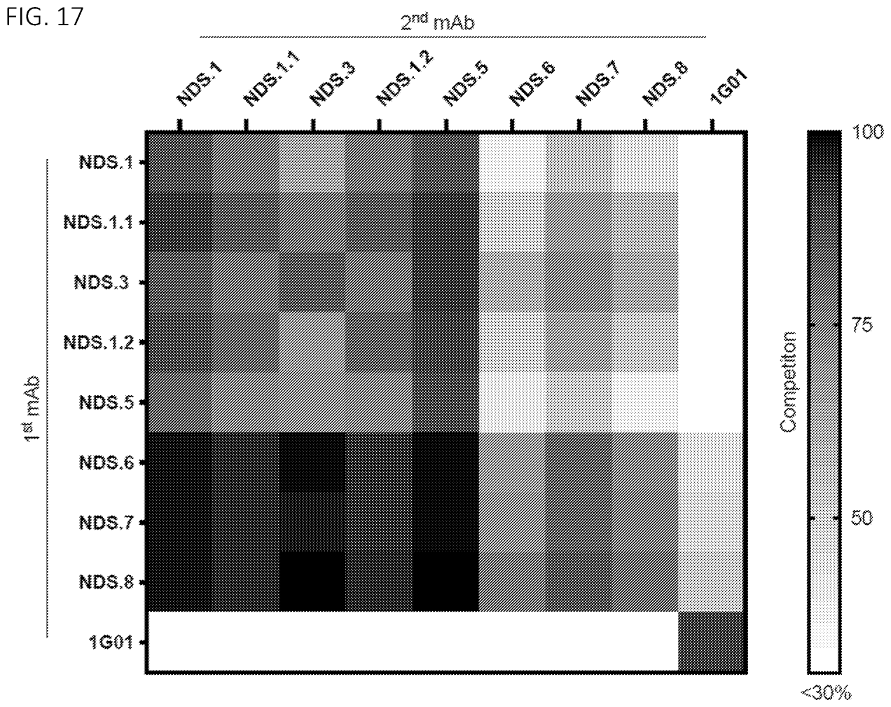

To characterize NA-directed B cell responses upon H3N2 virus infection in humans, peripheral blood mononuclear cells (PBMCs) were collected from two convalescent individuals who had a confirmed influenza A H3N2 infections in 2015 and 2021 (“donor A” and “donor B”, respectively). Antigen-specific B cells were single-cell sorted from PBMCs with N2 NA probes (FIGS. 7A-7C) and corresponding heavy and light chain variable genes were sequenced. A total of 44 and 66 paired immunoglobulin heavy and light chain gene sequences from donor A and donor B were collected, respectively, and analyzed for immunogenetic compositions (FIG. 1A). There were several clonal expansions of NA-specific immunoglobulin sequences in donor A samples likely associated with the recent H3N2 infection, whereas donor B had mostly unique sequences with only a few expanded clones. Representative immunoglobulin heavy and light chain genes were subcloned into human IgG1 backbone vectors and recombinant mAbs were produced in mammalian cells by transient transfection of corresponding heavy and light chain plasmids. Six mAbs (NDS.1, NDS.1.1, NDS.1.2, NDS.3, NDS.5, and NDS.6) were identified that bound to all four members of a panel of four N2 NAs from three seasonal H3N2 viruses and one swine-origin variant H3N2 (H3N2v) virus (FIG. 1B). Binding data for NDS.7 and NDS.8 is shown in FIG. 17. Overall, these mAbs bound equally well to the four tested N2 NAs with the exception of NDS.6, which showed lower binding to two out of four NAs. When these mAbs were compared to 1G01, the most well-characterized NA catalytic site-directed mAb (Stadlbauer et al., Science 366: 499-504 (2019)), the mAbs from this study exhibited equivalent or higher binding than 1G01 (FIG. 1B).

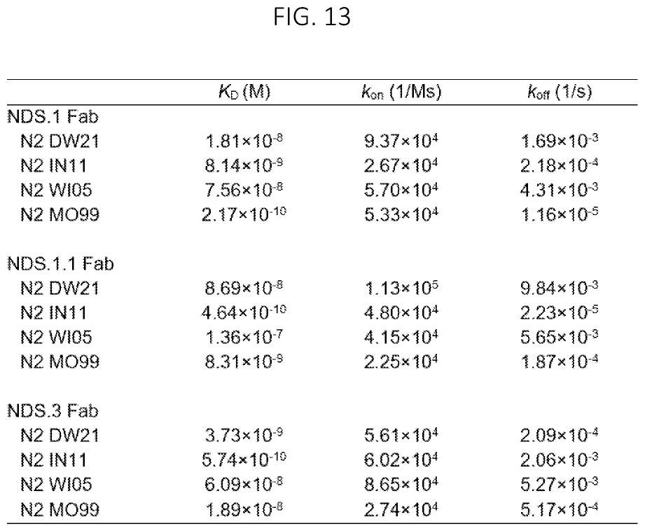

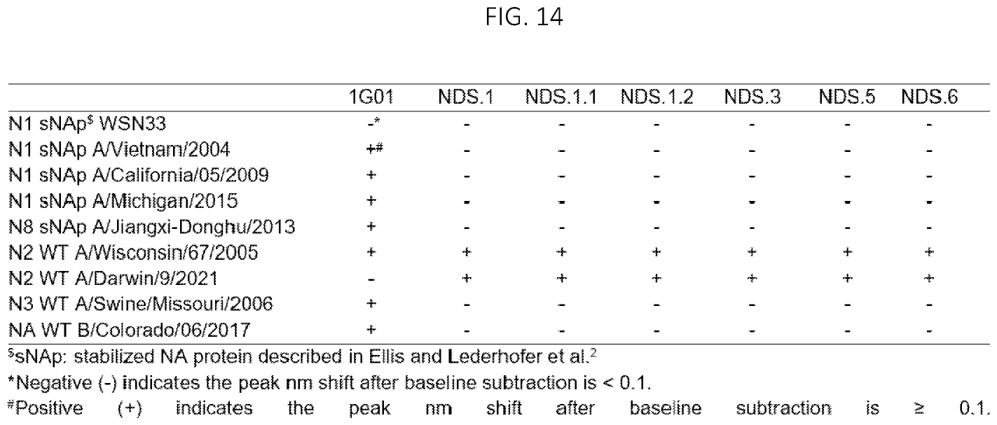

Next, the mAbs were tested for the ability to target the NA catalytic site and compete with 1G01 for NA binding. A binding competition assay by using biolayer interferometry (BLI) revealed that none of the mAbs (NDS.1, NDS.1.1, NDS.1.2, NDS.3, NDS.5, NDS.7 and NDS.8) showed any appreciable competition with 1G01, indicating that they recognize antigenic surfaces distinct from the conserved catalytic site (FIG. 1C, FIG. 17). To better understand their binding specificities, the binding kinetics of Fabs generated from the NDS.1, NDS.1.1, and NDS.3 to soluble recombinant N2 tetramers by BLI was measured (FIG. 1D; FIGS. 7A-7C; FIG. 13). Fabs of NDS.1 and NDS.3 showed sub-nanomolar to weaker nanomolar affinity to all tested NAs (FIG. 1D; FIGS. 7A-7C). The Fab of NDS.1.1 had lower overall affinity with a noticeably faster off-rate for A/Wisconsin/67/2005 (WI05) and A/Darwin/09/2021 (DW21) NAs compared to NDS.1 and NDS.3 Fabs. Both NDS.1.1 and NDS.3 Fabs showed the highest affinity to the NA of swine-origin H3N2v virus A/Indiana/10/2011 (IN11v). It was also confirmed that all tested mAbs were N2-specific and did not cross-react with other group 1 or group 2 NA subtypes (FIG. 14). In summary, six mAbs were isolated from H3N2-infected individuals that recognize conserved epitopes amongst N2 subtype NAs outside the catalytic site.

### Example 3: Cryo-EM Structures of Fabs NDS.1 and NDS.3 in Complex with NA

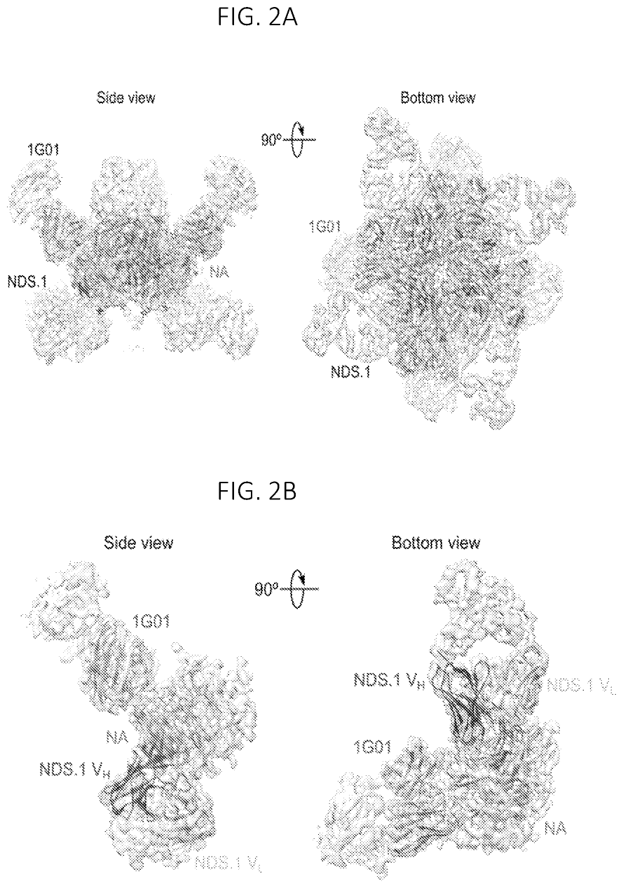

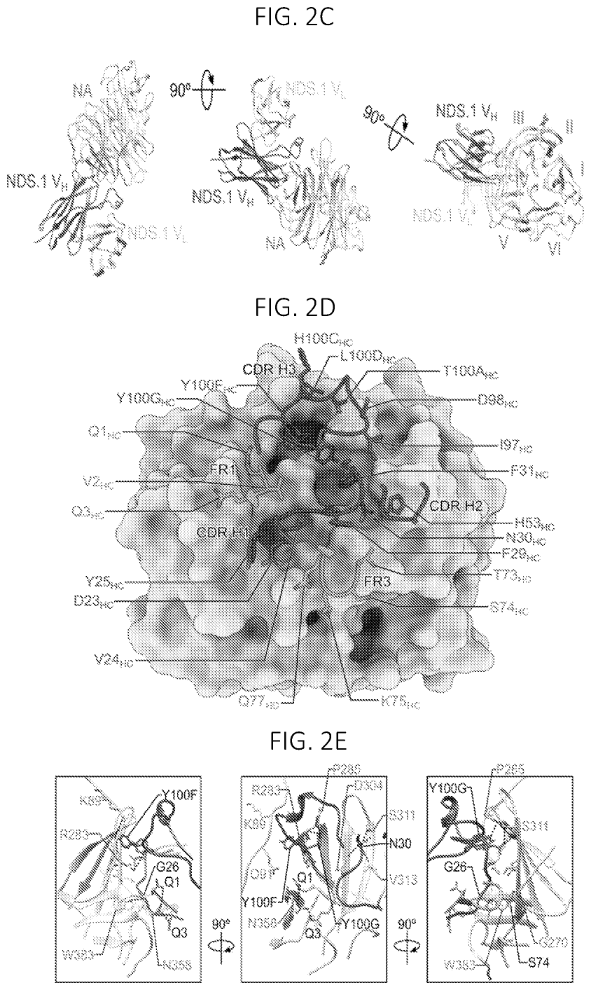

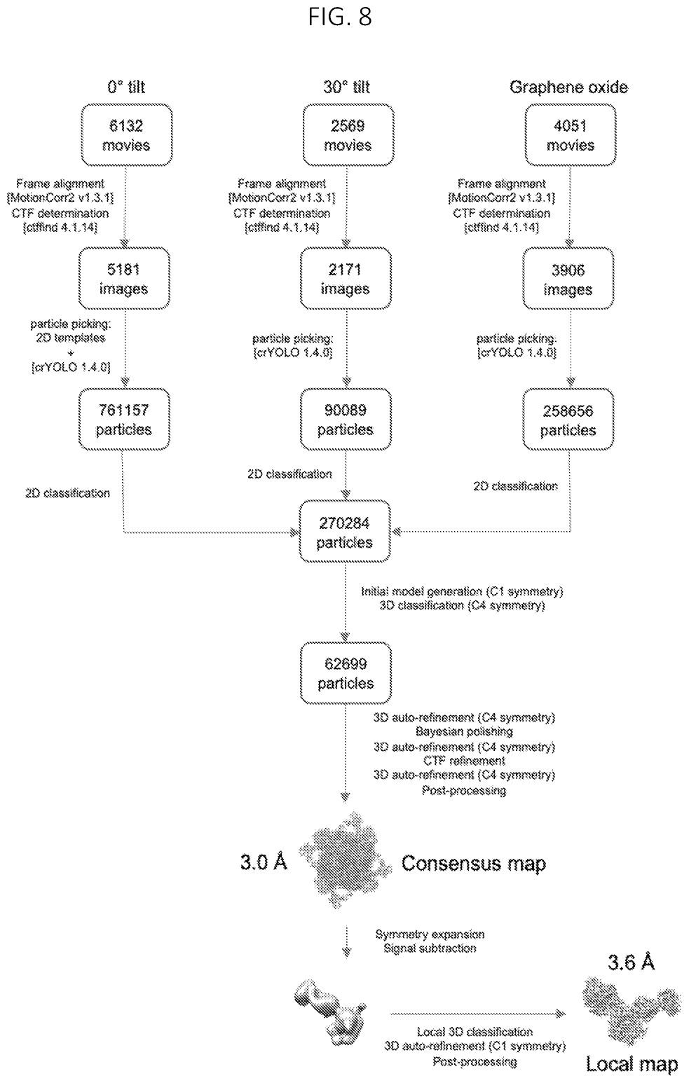

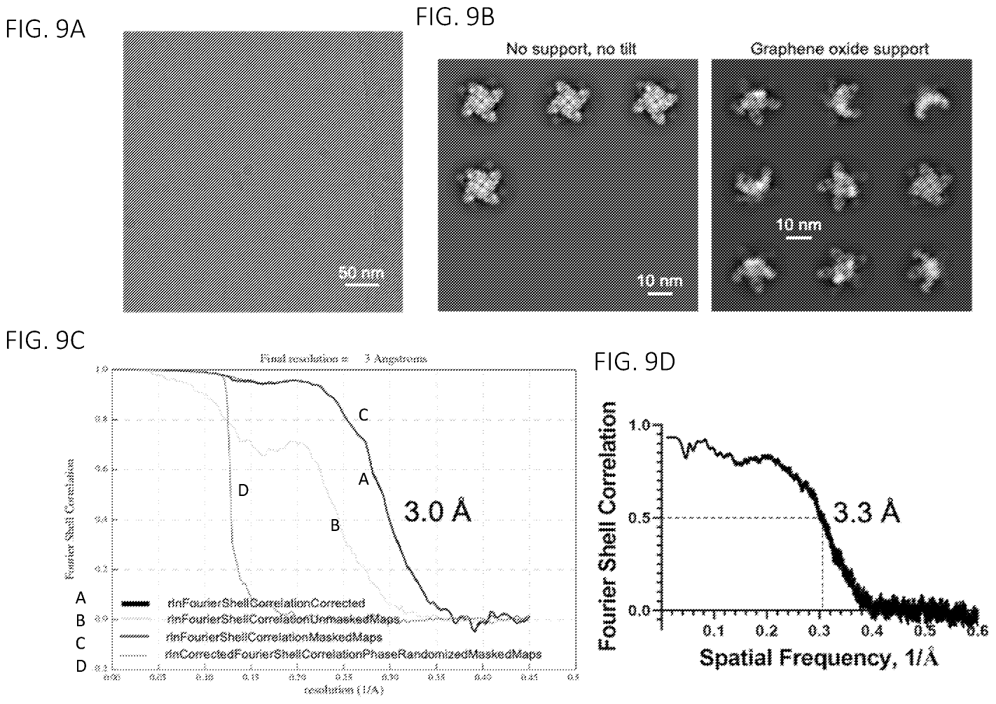

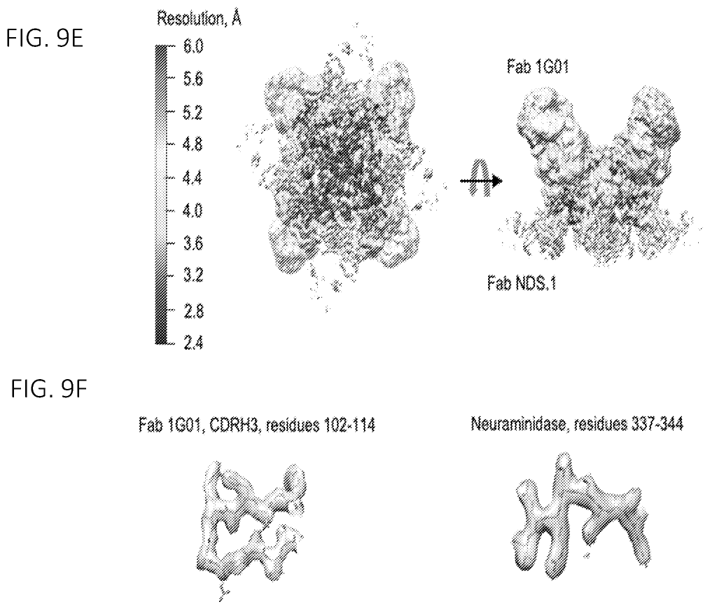

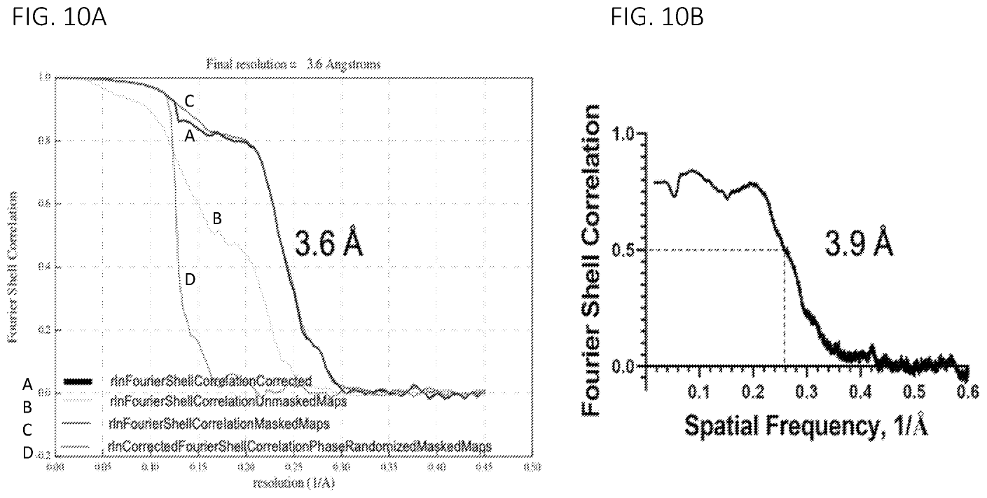

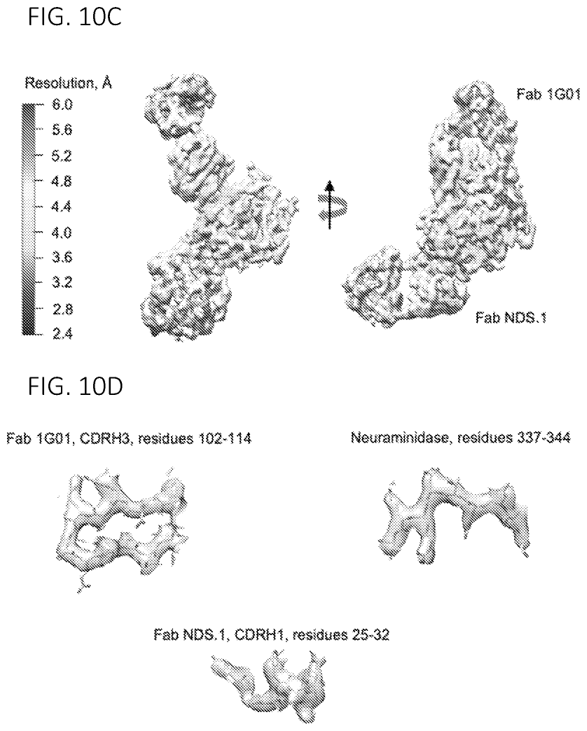

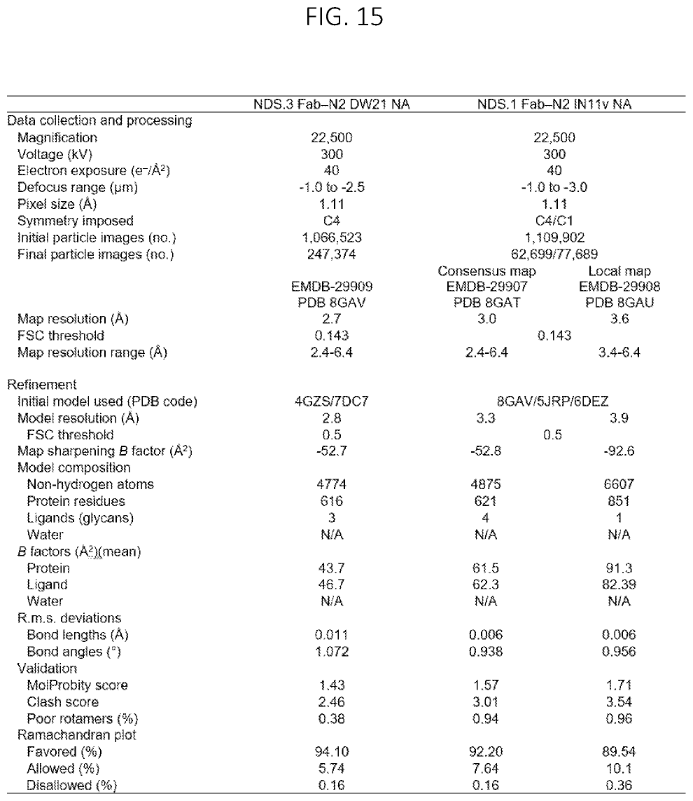

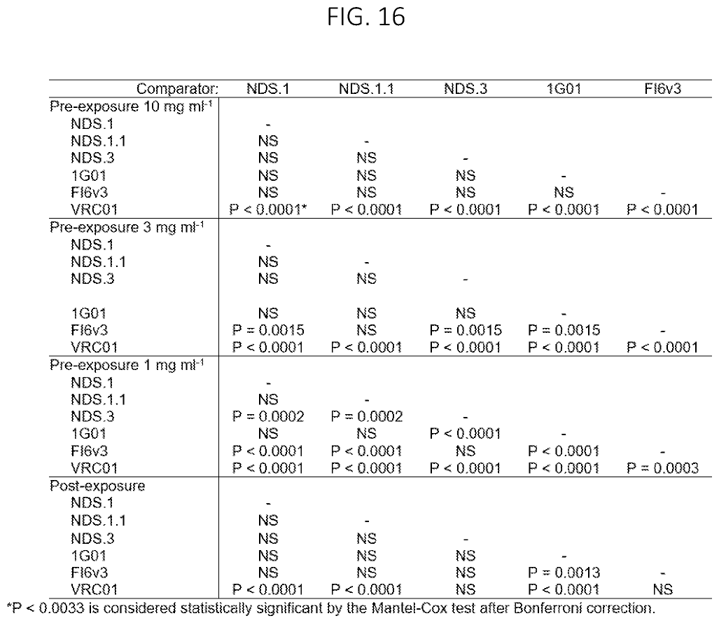

To identify the epitopes recognized by mAbs NDS.1 and NDS.3 and elucidate the structural basis of their broad N2 specificity, cryo-EM structures of Fab NDS.1 in complex with N2 IN11v NA and of Fab NDS.3 in complex with N2 DW21 NA were determined (FIG. 15). 1G01 (Stadlbauer et al., Science 366: 499-204 (2019)), which targets the catalytic site of NA from the top of the mushroom-shaped NA globular head, was added to the IN11v-NDS.1 complex to overcome the preferred orientation of particles in vitrified ice (FIG. 8; FIGS. 9A-9F). The 3.0 Å cryo-EM map of this ternary complex revealed that the NDS.1 and 1G01 Fabs bind on opposite sides of NA, with four NDS.1 Fabs bound to the underside of the NA globular head (FIG. 2A). Each NDS.1 Fab interacted with a single NA protomer and was positioned at a slight upward angle to the horizontal plane of the tetramer, with the constant region extending outward from under the head. The cryo-EM density for NDS.1 was weaker than the rest of the consensus map owing to partial Fab occupancy. To improve the NDS.1 density, symmetry expansion and signal subtraction outside a mask encompassing one NA protomer with the two bound Fabs was performed, which was followed by 3D classification within a mask encompassing the target Fab to isolate the subset of complexes with occupied NDS.1 Fab (FIG. 8; FIGS. 10A-10D). Subsequent local refinement produced a 3.6 Å map with strong NDS.1 density, which allowed us to build an atomic model of the NDS.1-NA complex (FIG. 2B-2C). Despite only the heavy chain of NDS.1 contributing to the interaction interface, the total buried surface areas on NA and the Fab were relatively large (1,014 and 1,118 Å², respectively). Of the six four-stranded β sheets forming the NA molecule (Jedrizejas et al., Biochemistry 34: 3144-3151 (1995)), NDS.1 targeted the bottom regions of β sheets IV and V, with some additional interactions with the N-terminus (β sheet VI; FIG. 2C). CDR H1 as well as a portion of the 17-residue long CDR H3 were positioned along a crevice in the underside surface of the NA protomer, with three bulky aromatic residues (Phe31, Tyr100F and Tyr100G), along with Ser28 and Asn30, filling the crevice (FIG. 2D). CDR H1 and CDR H3 contributed 378 and 386 Å², respectively, to the total buried surface area on the Fab, whereas the contribution of CDR H2 was minor (54 Å²). In addition to the CDRs, two framework regions (FRs) of the heavy chain interacted with NA, including residues 1-3 of FR H1 and residues 73-77 of FR H3 (FIG. 2D). These two regions flanked CDR H1, creating the remainder of the paratope, with buried surface areas of 179 A2 and 122 A2, respectively (total FRs contribution of 27%). In total, 14 hydrogen bonds between NDS.1 and IN11 NA were observed, involving side chain as well as main chain atoms on both sides (FIG. 2E).

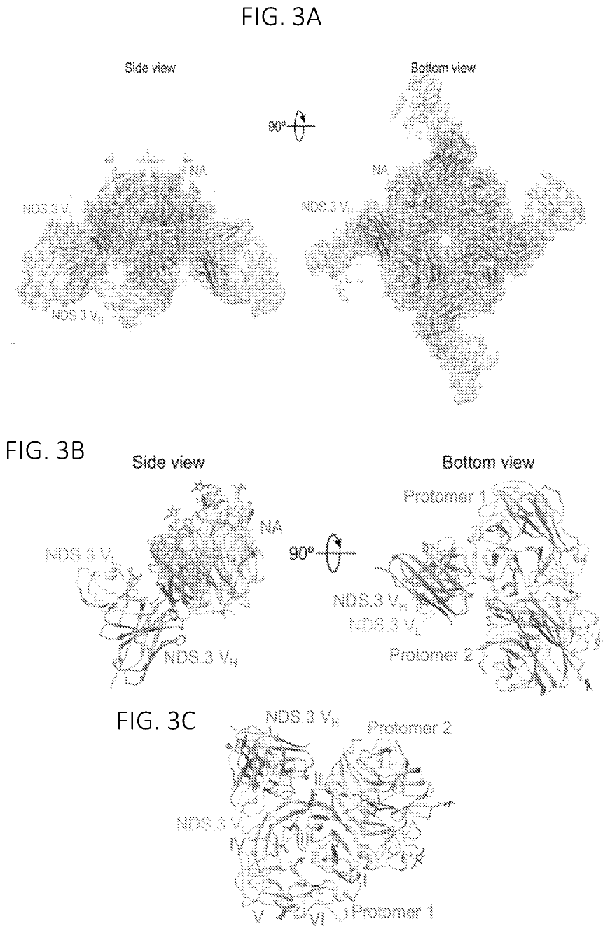

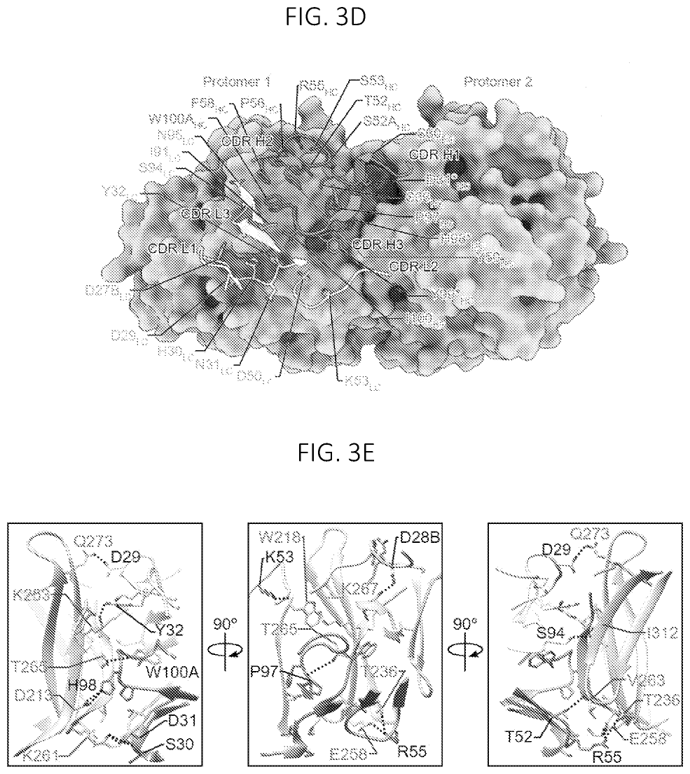

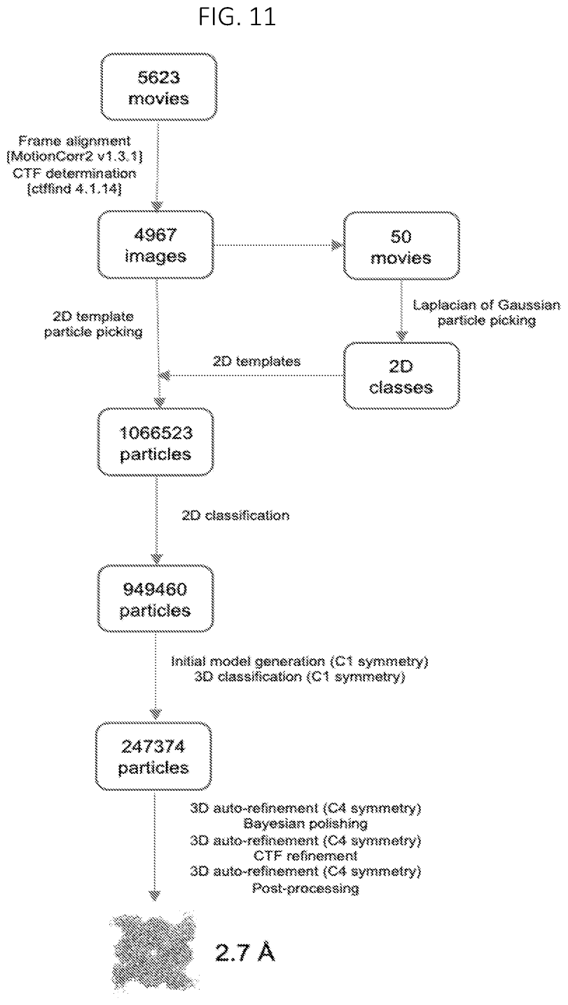

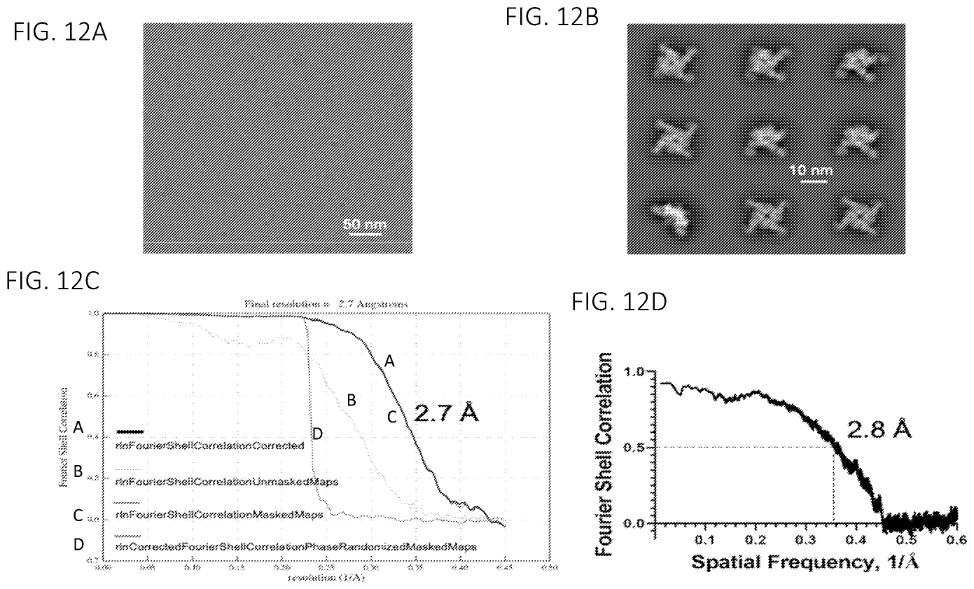

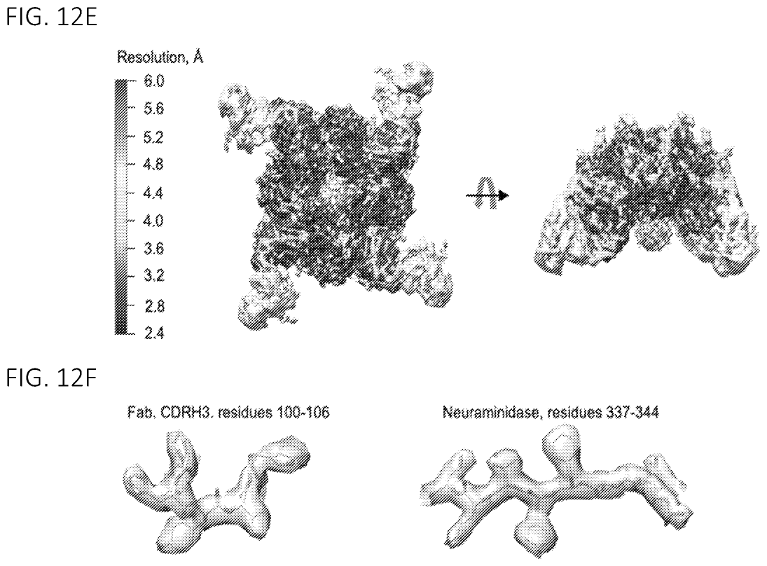

The cryo-EM structure of NDS.3 Fab in complex with DW21 NA was solved to a resolution of 2.7 Å (FIG. 3A; FIG. 11; FIGS. 12A-12E; FIG. 15). Fab NDS.3 also targeted the underside of the NA globular head, approaching NA upwards at an angle of about 30° to the horizontal plane of the tetramer (FIG. 3B). It interacted primarily with β sheet III of NA (FIG. 3C), as well as with one β strand each of β sheets II and IV that were adjacent to β sheet III. Each of the four bound Fabs contacted mainly the side and bottom regions of one NA protomer, burying 898 Å² and 933 Å² on the NA and Fab surfaces, respectively (FIG. 3A). CDR1 and CDR3 of the heavy chain were situated close to the inter-protomer interface of NA, resulting in residues Asp31, His98 and Tyr99 forming a small additional interface (buried surface areas on NA and Fab of 63 Å² and 64 Å², respectively) with a neighboring NA protomer (protomer 2) (FIGS. 3B-3D). All the CDRs of the heavy and light chains of NDS.3 participated in the interaction with the primary protomer 1, which involved multiple polar and charged contacts (FIG. 3D). The CDRs of the heavy chain accounted for the majority of the buried surface area on the NA (66%, 634 Å²) contributing four salt bridges (between Asp31^{HC} and Lys261, Arg55^{HC} and Glu258, His98^{HC} and Asp213) and seven hydrogen bonds to the interaction interface with protomer 1 (FIG. 3E). Furthermore, aromatic residues Tyr50 and Phe58 of CDR H2 and Tyr99 and Trp100A of CDR H3 engaged in hydrophobic contacts with NA residues and provided additional shape complementarity. The CDRs of the light chain formed one salt bridge (between Asp27B^{LC} and Lys267) and five hydrogen bonds, covering 328 Å² on NA protomer 1.

### Example 4: NDS.1 and NDS.3 Epitope Comparison and Conservation

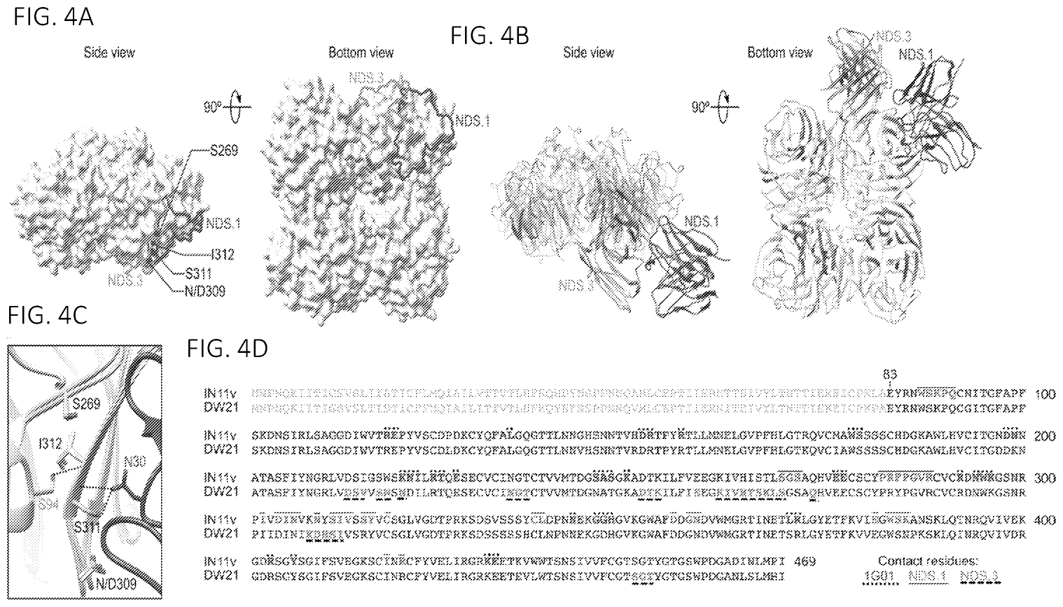

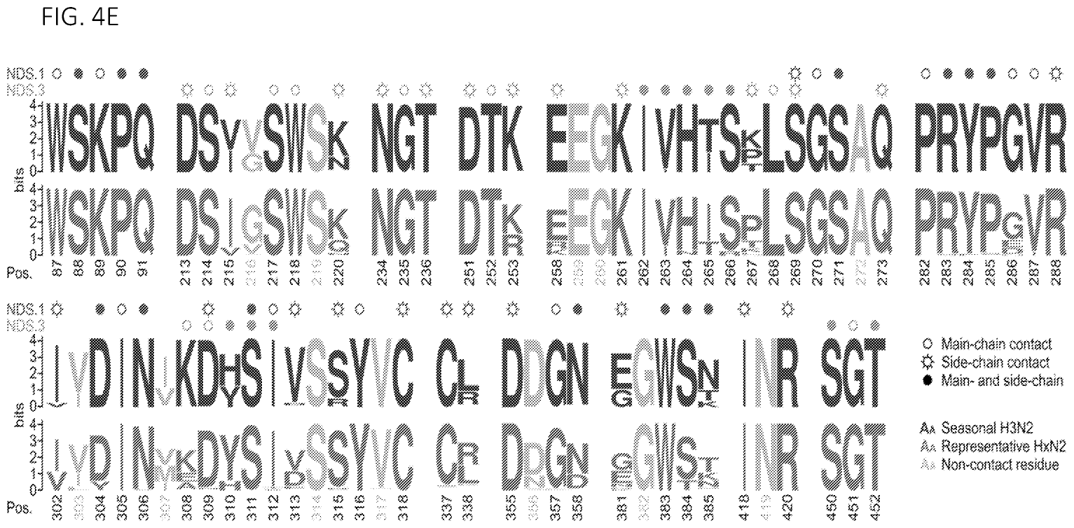

The structures of IN11 and DW21 NAs were highly similar to each other (root mean square deviation in Ca atom positions of 0.54 Å). This allowed for a comparison between the epitopes of NDS.1 and NDS.3 by superposing the two NA molecules. The two antibodies targeted different but adjacent areas on the NA underside, with their epitope outlines sharing a border (FIGS. 4A-4B). The four NA residues that contributed to both epitopes included Ser269, located in the loop connecting β sheets III and IV, as well as Asn309 (IN11v)/Asp309 (DW21), Ser311 and Ile312, belonging to β sheet IV. The contacts formed by these residues were mainly limited to Van der Waals interactions, with the exception of one hydrogen bond between the backbone nitrogen of Ile312 of NA and the side-chain oxygen of Ser94 of the NDS.3 light chain, and two hydrogen bonds between the side-chain nitrogen of Asn30 of the NDS.1 heavy chain and the backbone oxygen and side-chain oxygen of NA Ser311 (FIG. 4C). Of note, the four NA residues participating in both epitopes maintained the same positions in the two structures, and superposition of the IN11v/NDS.1 and DW21/NDS.3 complexes revealed no clashes between the two Fabs (FIG. 4B). Together, NDS.1 and NDS.3 epitopes cover a large surface on the NA underside, and the two epitopes are not overlapping (FIG. 4D).

The epitopes of NDS.1 and NDS.3 appeared well conserved amongst human seasonal H3N2 viruses spanning more than 50 years of antigenic evolution. The majority of the residues on NA contacting NDS.1 (33/37, 89%) and NDS3 (26/31, 84%) had virtually no variation among 188 non-redundant representative NA sequences of seasonal human H3N2 viruses (FIG. 4E). Substitution at position 267, one of the most variable residues within the NDS.3 epitope, from Lys (DW21) to Thr (IN11v), which is predicted to eliminate a salt bridge with Asp27B^{LC}, did not have a major impact on its recognition by NDS.3 (FIG. 1D). Interestingly, the epitopes of NDS.1 and NDS.3 are still well conserved (30/37, 81% and 25/31, 81% for NDS.1 and NDS.3, respectively) when we use 80 non-redundant representative N2 sequences of HxN2 viruses including H1N2, H2N2, H3N2, H7N2, and H9N2 (FIG. 4E). This suggests that the cross-reactivity of NDS.1 and NDS.3 may extend beyond H3N2 viruses. Overall, both the NDS.1 and NDS.3 epitopes are largely conserved among human H3N2 viruses and also across HxN2 viruses.

### Example 5: Biological Activity of NA Dark Side-Targeting Antibodies

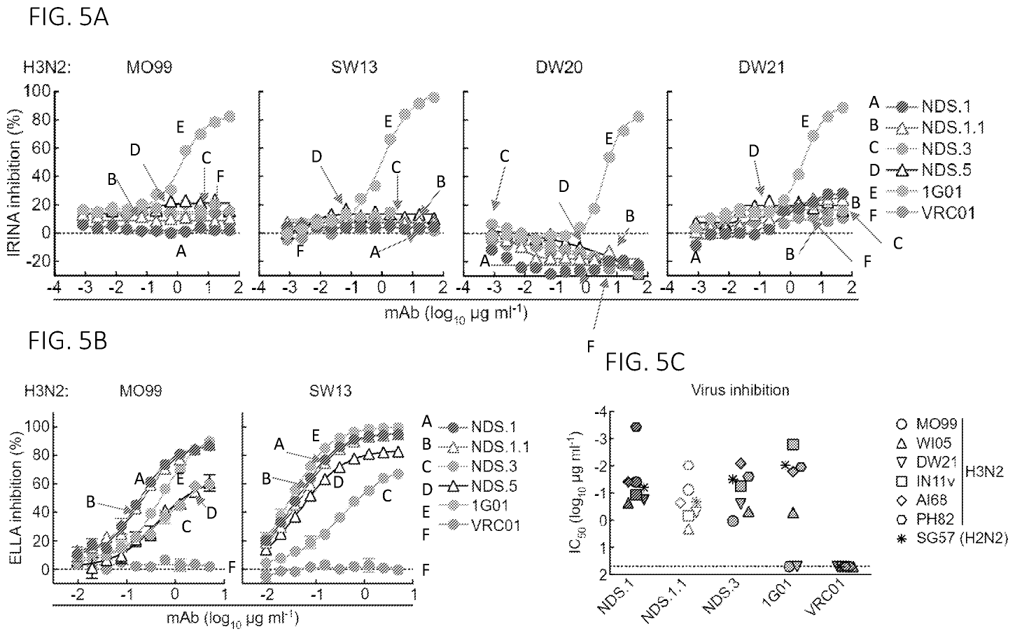

The functionality of the NA dark side-targeting antibodies using both recombinant NA proteins and viruses was assessed. It is known that the catalytic activity of NA can be blocked not only by directly targeting the catalytic pocket, but also through steric hindrance by antibody binding to nearby sites on NA or even the stem of neighboring HA molecules (Kosik et al., J. Exp Med. 216: 304-316 (2019)). The NA inhibitory activity of the dark side-targeting antibodies was first measured by IRINA (Influenza Replication Inhibition Neuraminidase-based Assay) (Patel et al., Antiviral Res. 208: 105457 (2022)), which detects the enzymatic activity of NA expressed on the surface of influenza infected cells. The IRINA assay is a direct, functional enzymatic assay that measures NA catalytic activity through the release of the fluorogenic end product 4-methylumbelliferone from the non-fluorescent small molecule substrate MUNANA (2′-(4-methylumbelliferyl)-α-D-N-acetylneuraminic acid). NA inhibition (NAI) is therefore only observed in the NA-star assay for antibodies that directly compete with substrate binding to the catalytic site (Potier et al., Anal. Biochem 94: 287-296 (1979)). As expected, while the catalytic site-directed 1G01 showed NAI activity in this assay, all of our mAbs targeting the NA dark side showed no NAI activity to four H3N2 viruses tested (FIG. 5A). To better understand the biological relevance of the dark side-targeting antibodies, the enzyme-linked lectin assay (ELLA) was performed. ELLA measures the cleavage of sialic acid from the glycoprotein fetuin by NA, which can be inhibited by antibodies not only binding directly to the catalytic site but also through steric hindrance, unlike the NA-star assay (Lambré et al., J ImmunoL Methods 135:49-57 (1990)). All the dark side-directed mAbs showed NAI activity comparable to the control antibody 1G01 to both H3N2 viruses tested in ELLA (FIG. 5B). This demonstrates that the NA dark side-directed mAbs are able to inhibit NA catalytic activity by sterically hindering access to glycoprotein substrates.

While NA-directed antibodies do not typically block viral entry to host cells, they inhibit viral egress of nascent particles from the infected cells through inhibition of NA catalytic activity (Krammer et al., M Bio 9: c02332-17 (2018); Giurgea et al., Vaccines 8 (2020)). Therefore, the ability of NA dark side-directed mAbs to inhibit in vitro viral growth of five H3N2, one H3N2v, and one H2N2 viruses was assessed. The virus growth inhibition assay was performed using reporter influenza viruses in order to detect infected cells over time (Creanga et al. Nat Commun 12: 1722 (2021)). In this assay, mAbs were first incubated with viruses prior to adding to the substrate cells. The virus inoculum was removed after 4-5 hours and cells were kept in culture media supplemented with mAbs for another 24-28 hours. In order to calculate viral growth inhibition, the data was normalized it to inhibition by the NA inhibitor zanamivir (100% inhibition). All NA dark side-directed mAbs inhibited the growth of all viruses tested, including non-circulating H3N2v and H2N2 viruses (FIG. 5C). Interestingly, the control antibody 1G01 did not inhibit growth of the MO99 and DW21 H3N2 viruses, whereas all dark side-directed mAbs potently inhibited those viruses. Based on these results, the NA dark side-directed mAbs are capable of inhibiting not only a wide range of H3N2 viruses, but also divergent H3N2v and H2N2 viruses, by targeting highly conserved epitopes on the NA underside.

### Example 6: NA Dark Side-Directed mAbs Confer Protection Against Lethal H3N2 Influenza Virus Challenge in Mice

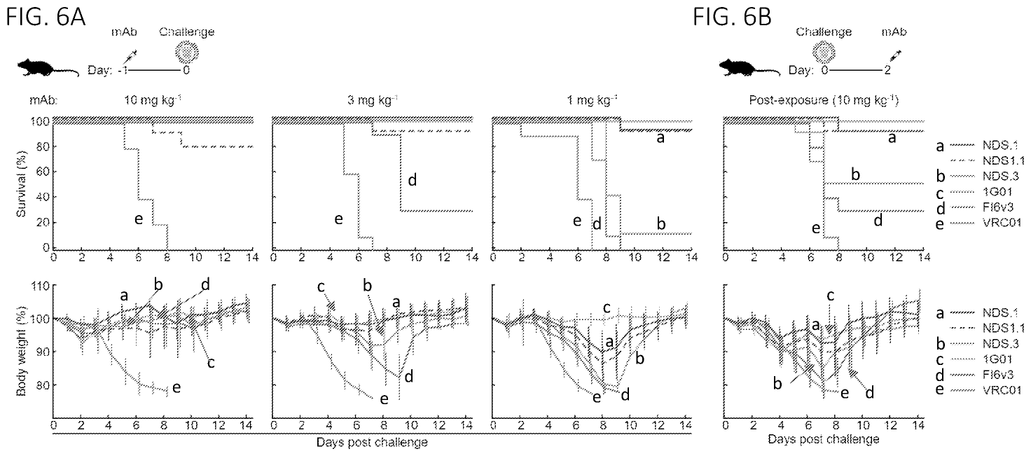

To see if the in vitro inhibitory activity of the NA dark side-directed mAbs can translate into in vivo efficacy, a series of lethal influenza virus challenge studies in mice were conducted. First, protective efficacy in a prophylactic setting where the mAbs were given 24 hours prior to challenge with A/Philippines/2/1982 (H3N2) virus (10×50% lethal dose (LD₅₀)) was measured. In this model, NDS.1 conferred full protection without any noticeable body weight loss when 10 and 3 mg kg⁻¹ were administered and afforded 90% survival when given the lowest dose of 1 mg kg⁻¹ (FIG. 6A). NDS.1.1 showed similar performance as NDS.1, providing 80-90% protection with all doses tested. NDS.3 conferred full protection with a dose of 10 and 3 mg kg⁻¹, although there was ˜8% mean body weight loss at days 8 and 9 when given 3 mg kg⁻¹. However, this mAb afforded only 10% survival when the lowest amount (1 mg kg⁻¹) was administered (FIG. 6A). Consistent with a previous report (Stadlbauer et al., Science 366: 499-504 (2019)), the catalytic site-directed 1G01 conferred full protection at all doses tested. The control anti-HA stem mAb FI6v3⁴¹ provided weaker protection compared to all tested anti-NA mAbs. Full protection was observed when the highest 10 mg kg⁻¹ of FI6v3 was given, but protective efficacy rapidly dropped as the mAb dose decreased (FIG. 6A). Overall, NA dark side-directed mAbs provided potent protection from mortality and morbidity caused by H3N2 virus infection in mice when prophylactically administered.

Since commercial antivirals targeting influenza virus NA are efficacious as therapeutics after symptom onset, the protective efficacy of the dark side-directed mAbs in a post-exposure therapeutic setting was assessed. Mice were infected with a 5×LD₅₀ of A/Philippines/2/1982 (H3N2) virus 48 hours prior to administration of mAbs at 10 mg kg⁻¹. In this setting, mice did not show appreciable body weight loss at the time of mAb administration. Ninety percent of mice receiving either NDS.1 or NDS.1.1 survived, with a mild body weight loss of ˜10% which was similar to the control 1G01-treated mice, whereas NDS.3-treated mice resulted in 50% survival with more severe body weight loss (FIG. 6B). The control anti-HA mAb FI6v3 afforded only partial protection (30% survival) in this therapeutic setting. The NA dark side-directed mAbs were highly protective not only when given as pre-exposure prophylaxis even at a dose as low as 1 mg kg⁻¹, but also as post-exposure therapeutics in a mouse model.

Influenza virus NA has long been central for drug discovery efforts, with licensed antivirals targeting NA continuing to save countless lives since their first approval in 1999. Although it has been known that antibodies can inhibit the catalytic activity of NA since the 1980s, only a few mAbs have been identified in humans to date that have activity similar to commercial NA inhibitors (Stadlbauer et al., Science 366: 499-504 (2019); Yasuhara et al., Nat. Commun. 13: 6602 (2020)). Besides these rare mAbs, antibodies to antigenic sites peripheral to the catalytic site can also inhibit NA activity by sterically hindering access to sialoside substrates, but those antibodies are generally less cross-reactive (Wohlbold et al., Nat. Microbiol. 2: 1415-1424 (2017)). Antibodies such as CD6 and NA-22 recognize the lateral surface of the NA globular head distant from its catalytic site, yet still inhibit the catalytic activity of NA through indirect steric hindrance (Wan et al., Nat. Commun. 6: 6114 (2015); Zhu et al., Cell Host Microbe 26: 729-738; Gilchuk et al., Cell Host Microbe 26: 715-728 (2019)). Finally, a few reports have noted that the catalytic activity of NA can also be inhibited through anti-HA stem antibodies through steric hindrance (Kosik et al., J. Exp. Med. 216: 304-316 (2019); Chen et al., J. Virol 93 (2019)). Nevertheless, the understanding of the NA antigenic landscape is still far from complete.

B cell lineages specific to the NA dark side responded to the recent H3N2 virus infection in a donor, and they cross-react with N2 NA from viruses spanning more than six decades. The dark side-directed mAbs can inhibit the catalytic activity of NA on the virus when a macromolecular substrate (i.e., fetuin) was used, likely through indirect steric hindrance, and blocked virus propagation and spread in vitro through inhibition of its sialidase activity. These viral inhibitory properties of the dark side-directed mAbs are analogous to the NA inhibitors such as oseltamivir and zanamivir or the catalytic site-directed antibodies such as 1G01 and DA03E17 (Stablbuer et al., Science 366: 499-504 (2019); Yasuhara et al., Nat Commun 13: 6602 (2022)), and they all can confer both pre- and post-exposure prophylactic protection in murine infection model of influenza. Additionally, the hallmark substitutions associated with resistance to oseltamivir and/or zanamivir such as E119V and 1222L (Wu et al., J. Virol. 90: 10693-10700 (2016)) does not impact the dark side epitopes.

1. An isolated monoclonal antibody or antigen binding fragment thereof, comprising:
a heavy chain variable region (V_{H}) and a light chain variable region (V_{L}) comprising a heavy chain complementarity determining region (HCDR)1, a HCDR2, and a HCDR3 and a light chain complementarity determining region (LCDR)1, a LCDR2, and a LCDR3, wherein the V_{H} and V_{L} are set forth as one of:
a) SEQ ID NOs: 1 and 5, respectively (NDS.1);
b) SEQ ID NOs: 17 and 21, respectively (NDS.3);
c) SEQ ID NOs: 9 and 13, respectively (NDS.1.1);
d) SEQ ID NOs: 25 and 29, respectively (NDS.1.2);
e) SEQ ID NOs: 33 and 37, respectively (NDS.6);
f) SEQ ID NOs: 41 and 45, respectively (NDS.8);
g) SEQ ID NOs: 49 and 53, respectively (NDS.5); or
h) SEQ ID NOs: 57 and 61, respectively (NDS.7);
wherein the monoclonal antibody specifically binds to influenza A neuraminidase (NA) protein and inhibits an N2 influenza A virus.

2. The isolated monoclonal antibody or antigen binding fragment of claim 1, wherein:
a) the HCDR1, the HCDR2, the HCDR3, the LCDR1, the LCDR2, and the LCDR3 comprise the amino acids sequences set forth as SEQ ID NOs: 2, 3, 4, 6, 7, and 8, respectively;
b) the HCDR1, the HCDR2, the HCDR3, the LCDR1, the LCDR2, and the LCDR3 comprise the amino acids sequences set forth as SEQ ID NOs: 18, 19, 20, 22, 23, and 24 respectively;
c) the HCDR1, the HCDR2, the HCDR3, the LCDR1, the LCDR2, and the LCDR3 comprise the amino acids sequences set forth as SEQ ID NOs: 10, 11, 12, 14, 15, and 16, respectively;
d) the HCDR1, the HCDR2, the HCDR3, the LCDR1, the LCDR2, and the LCDR3 comprise the amino acids sequences set forth as SEQ ID NOs: 26, 27, 28, 30, 31, and 32 respectively;
e) the HCDR1, the HCDR2, the HCDR3, the LCDR1, the LCDR2, and the LCDR3 comprise the amino acids sequences set forth as SEQ ID NOs: 34, 35, 36, 38, 39, and 40 respectively;
f) the HCDR1, the HCDR2, the HCDR3, the LCDR1, the LCDR2, and the LCDR3 comprise the amino acids sequences set forth as SEQ ID NOs: 42, 43, 44, 46, 47, and 48, respectively;
g) the HCDR1, the HCDR2, the HCDR3, the LCDR1, the LCDR2, and the LCDR3 comprise the amino acids sequences set forth as SEQ ID NOs: 50, 51, 52, 54, 55, and 56, respectively; or
h) the HCDR1, the HCDR2, the HCDR3, the LCDR1, the LCDR2, and the LCDR3 comprise the amino acids sequences set forth as SEQ ID NOs: 58, 59, 60, 62, 63, and 64, respectively.

3. The isolated monoclonal antibody or antigen binding fragment of claim 1, wherein
a) the V_{H} and the V_{L} comprise the amino acid sequences at least 90% identical to the amino acid sequences set forth as SEQ ID NOs: 1 and 5, respectively;
b) the V_{H} and the V_{L} comprise the amino acid sequences at least 90% identical to the amino acid sequences set forth as SEQ ID NOs: 17 and 21, respectively;
c) the V_{H} and the V_{L} comprise the amino acid sequences at least 90% identical to the amino acid sequences set forth as SEQ ID NOs: 9 and 13, respectively;
d) the V_{H} and the V_{L} comprise the amino acid sequences at least 90% identical to the amino acid sequences set forth as SEQ ID NOs: 25 and 29, respectively;
e) the V_{H} and the V_{L} comprise the amino acid sequences at least 90% identical to the amino acid sequences set forth as SEQ ID NOs: 33 and 37, respectively;
f) the V_{H} and the V_{L} comprise the amino acid sequences at least 90% identical to the amino acid sequences set forth as SEQ ID NOs: 41 and 45, respectively;
g) the V_{H} and the V_{L} comprise the amino acid sequences at least 90% identical to the amino acid sequences set forth as SEQ ID NOs: 49 and 53, respectively; or
h) the V_{H} and the V_{L} comprise the amino acid sequences at least 90% identical to the amino acid sequences set forth as SEQ ID NOs: 57 and 61, respectively.

4. The isolated monoclonal antibody or antigen binding fragment of claim 1, comprising a human framework region.

5. The isolated monoclonal antibody or antigen binding fragment of claim 1, wherein:
a) the V_{H} and the V_{L} comprise the amino acid sequences set forth as SEQ ID NOs: 1 and 5, respectively;
b) the V_{H} and the V_{L} comprise the amino acid sequences set forth as SEQ ID NOs: 17 and 21, respectively;
c) the V_{H} and the V_{L} comprise the amino acid sequences set forth as SEQ ID NOs: 9 and 13, respectively;
d) the V_{H} and the V_{L} comprise the amino acid sequences set forth as SEQ ID NOs: 25 and 29, respectively;
e) the V_{H} and the V_{L} comprise the amino acid sequences set forth as SEQ ID NOs: 33 and 37, respectively;
f) the V_{H} and the V_{L} comprise the amino acid sequences set forth as SEQ ID NOs: 41 and 45, respectively;
g) the V_{H} and the V_{L} comprise the amino acid set forth as SEQ ID NOs: 49 and 53, respectively; or
h) the V_{H} and the V_{L} comprise the amino acid sequences set forth as SEQ ID NOs: 57 and 61, respectively.

6. The isolated monoclonal antibody of claim 1, wherein the antibody comprises a human constant domain.

7. The isolated monoclonal antibody or antigen binding fragment of claim 1, wherein the antibody is a human antibody.

8. The isolated monoclonal antibody of claim 1, wherein the antibody is an IgG.

9. The isolated monoclonal antibody of claim 1, comprising a recombinant constant domain comprising a modification that increases the half-life of the antibody.

10. The isolated monoclonal antibody of claim 9, wherein the modification increases binding to the neonatal Fc receptor.

11. The antigen binding fragment of claim 1.

12. The antigen binding fragment of claim 11, wherein the antigen binding fragment is a Fv, Fab, F(ab′)₂, scFV or a scFV₂ fragment.

13. The isolated monoclonal antibody or antigen binding fragment of claim 1, wherein the influenza virus is an H3N2 influenza virus.

14. The isolated monoclonal antibody or antigen binding fragment of any one of claim 1, conjugated to a detectable marker.

15. A bispecific antibody comprising the monoclonal antibody or antigen binding fragment of claim 1.

16. An isolated nucleic acid molecule encoding the monoclonal antibody or antigen binding fragment of claim 1, or a bispecific antibody comprising the antibody or antigen binding fragment.

17. The nucleic acid molecule of claim 16, wherein the nucleic acid molecule is a cDNA sequence encoding the V_{H} or V_{L}.

18. The nucleic acid molecule of claim 17, wherein the nucleic acid molecule is a cDNA sequence encoding the V_{H} and/or V_{L} of an scFv antigen binding fragment.

19. The nucleic acid molecule of claim 16, operably linked to a promoter.

20. A vector comprising the nucleic acid molecule of any claim 16.

21. A host cell comprising the nucleic acid molecule of claim 16 or vector comprising the nucleic acid molecule.

22. A pharmaceutical composition comprising an effective amount of the antibody or antigen binding fragment of claim 1, a bispecific antibody comprising the antibody or antigen binding fragment, a nucleic acid molecule encoding the antibody, antigen binding fragment or bispecific antibody, or a vector comprising the nucleic acid molecule; and
a pharmaceutically acceptable carrier.

23. (canceled)

24. A method of producing an antibody or antigen binding fragment that specifically binds to an influenza virus, comprising:
expressing one or more nucleic acid molecules encoding the antibody or antigen binding fragment of claim 1, or a bispecific antibody comprising the antibody or antigen binding fragment in a host cell; and
purifying the antibody, antigen binding fragment, or bispecific antibody from the host cell.

25-26. (canceled)

27. A method of inhibiting an influenza virus infection in a subject, comprising administering an effective amount of the antibody or antigen binding fragment of claim 1, a nucleic acid molecule encoding the antibody or antigen binding fragment, a vector comprising the nucleic acid molecule, or pharmaceutical composition comprising the antibody, antigen binding fragment, nucleic acid molecule or vector 23 to the subject, wherein the subject has or is at risk of the influenza virus infection

28. (canceled)

29. A method of identifying a protein as a vaccine comprising:
contacting the protein with the monoclonal antibody or antigen binding fragment of claim 1 under conditions sufficient to form an immune complex; and
detecting the presence of an immune complex,
wherein the presence of the immune complex indicates that the protein is a vaccine for an influenza virus infection.

30-34. (canceled)


SEQUENCE LISTING (66 sequences)

SEQ ID NO: 1 - protein, 125 aa, Homo sapiens
QVQIQQWGAGLLKPSDTLSLTCSVYGGSFNFYYWTWVRQPPGKGLEWLAEINHSGSINYIPSLNSRVALSVDTSKGQFSLRLSSVTAADTAVYYCTGVPIDYESLHLGYYPMDVWGQGTPVIVSS

SEQ ID NO: 2 - protein, 8 aa, Homo sapiens
GGSFNFYY

SEQ ID NO: 3 - protein, 7 aa, Homo sapiens
INHSGSI

SEQ ID NO: 4 - protein, 19 aa, Homo sapiens
TGVPIDYESLHLGYYPMDV

SEQ ID NO: 5 - protein, 107 aa, Homo sapiens
DIQMTQSPSSLSASVGDRVTITCRASQGISNYLAWWQQKPGQVPKLLIYAASTLQSGVPSRFSGSGSGTDFSLTISSLQPDDVAIYYCQKYNSAPRTFGQGTKVEIK

SEQ ID NO: 6 - protein, 6 aa, Homo sapiens
QGISNY

SEQ ID NO: 7 - skipped

SEQ ID NO: 8 - protein, 9 aa, Homo sapiens
QKYNSAPRT

SEQ ID NO: 9 - protein, 125 aa, Homo sapiens
QVQLRQWGAGLLKPSETLSLTCDVYGGSFNFYFWTWIRQPPGKGLEWLGEITHSGSATYIPSLKSRVTLSVDTSKGQFSLKLTSVNVADTAVYYCAGLPIDYDTVHLGYYPMDVWGQGTPVTVSS

SEQ ID NO: 10 - protein, 8 aa, Homo sapiens
GGSFNFYF

SEQ ID NO: 11 - protein, 7 aa, Homo sapiens
ITHSGSA

SEQ ID NO: 12 - protein, 19 aa, Homo sapiens
AGLPIDYDTVHLGYYPMDV

SEQ ID NO: 13 - protein, 107 aa, Homo sapiens
DIQMTQSPSSLSASVGDRVTITCRASQGIRNYLAWYQQKPGKVPKLLIYGASTLQSGVPSRFSGSGSGTDFSLTISSLQPEDIATYFCQEYNNAPRTFGQGTKVEIK

SEQ ID NO: 14 - protein, 6 aa, Homo sapiens
QGIRNY

SEQ ID NO: 15 - skipped

SEQ ID NO: 16 - protein, 9 aa, Homo sapiens
QEYNNAPRT

SEQ ID NO: 17 - protein, 122 aa, Homo sapiens [sequence omitted]

SEQ ID NO: 18 - protein, 8 aa, Homo sapiens
GFTFSDYS

SEQ ID NO: 19 - protein, 8 aa, Homo sapiens
TTSSGRPI

SEQ ID NO: 20 - protein, 15 aa, Homo sapiens
ARGDPHYIWGTYLDS

SEQ ID NO: 21 - protein, 110 aa, Homo sapiens
QSALTQPPSASGSPGQSVTISCTGTTSDFGDHNYVSWYQQRPGEAPKLIIYDVSKRPSGVPDRFSGSKSGNTASLTVSRLQADDEANYYCSSIEGSNTLLFGGGTKLTVL

SEQ ID NO: 22 - protein, 9 aa, Homo sapiens
TSDFGDHNY

SEQ ID NO: 23 - skipped

SEQ ID NO: 24 - protein, 10 aa, Homo sapiens
SSIEGSNTLL

SEQ ID NO: 25 - protein, 125 aa, Homo sapiens
QVQLQQWGAGLLKPSETLSLTCDVYGGAFNFYYWTWIRQSPGRGLEWLAEITHSGSATYTPSLKSRLTLAVDTSKGQFSLKLTSVTAADTAVYYCAALPIDYDTIYLGYYTMDVWGQGTPVAVSS

SEQ ID NO: 26 - protein, 8 aa, Homo sapiens
GGAFNFYY

SEQ ID NO: 27 - protein, 7 aa, Homo sapiens
ITHSGSA

SEQ ID NO: 28 - protein, 19 aa, Homo sapiens
AALPIDYDTIYLGYYTMDV

SEQ ID NO: 29 - protein, 107 aa, Homo sapiens
DIQMTQSPSSLSASLGDRVTITCRASQGISSYLAWYQQRPGKVPKLLIYAASTLQSGVSPRFSGSGSGTDFSLTISSLQPEDVAIYFCQEYDSAPRTFGQGTKVEIK

SEQ ID NO: 30 - protein, 6 aa, Homo sapiens
QGISSY

SEQ ID NO: 31 - skipped

SEQ ID NO: 32 - protein, 9 aa, Homo sapiens
QEYDSAPRT

SEQ ID NO: 33 - protein, 124 aa, Homo sapiens
QVQLVQSGPEVKKPGASVKVSCKASGYNFNRYGLTWVRQAPGQGLEWMGWISTYNSDTIYAREFRGRVTLTTDTSTRTAYMELRSLISADTAVYYCARDNVEEAFPISYYFDHWGQGTLVSVSS

SEQ ID NO: 34 - protein, 8 aa, Homo sapiens
GYNFNRYG

SEQ ID NO: 35 - protein, 8 aa, Homo sapiens
ISTYNSDT

SEQ ID NO: 36 - protein, 17 aa, Homo sapiens
ARDNVEEAFPISYYFDH

SEQ ID NO: 37 - protein, 110 aa, Homo sapiens
QSALTQPRSVSGSPGQSVTISCTGTSSDVGLYNYVSWYQHHPGKAPKFLIYDVTKRPSGVPDRFSGSKSGNTASLTISGLQAEDEADYYCCSYVGSYILVFGGGTKVTVL

SEQ ID NO: 38 - protein, 9 aa, Homo sapiens
SSDVGLYNY

SEQ ID NO: 39 - skipped

SEQ ID NO: 40 - protein, 10 aa, Homo sapiens
CSYVGSYILV

SEQ ID NO: 41 - protein, 119 aa, Homo sapiens
EVQLVESGGGLVKPGGSLRLSCAASGFTFSTYTMNWVRQAPGKGLEWVSSISSSGNYMYYADSVKGRFTISRDSAKNSLSLQMTSLSAEDTAVYYCARDKWIWDAFDIWGQGTMVTVSS

SEQ ID NO: 42 - protein, 8 aa, Homo sapiens
GFTFSTYT

SEQ ID NO: 43 - protein, 8 aa, Homo sapiens
ISSSGNYM

SEQ ID NO: 44 - protein, 12 aa, Homo sapiens
ARDKWIWDAFDI

SEQ ID NO: 45 - protein, 105 aa, Homo sapiens
FMTQSPFSLSVSPGERATLSCRASQSVRSKLAWYQQKPGQAPRLLISGASTRATGVPPRFSGSGSGTEFTFTISSLQSEDFAVYYCQQYNDWPRTFGQGTRVEIK

SEQ ID NO: 46 - protein, 6 aa, Homo sapiens
QSVRSK

SEQ ID NO: 47 - skipped

SEQ ID NO: 48 - protein, 9 aa, Homo sapiens
QQYNDWPRT

SEQ ID NO: 49 - protein, 120 aa, Homo sapiens
QLRLQESGPGLVKPSETLSLTCTVSGGSITSSPYYWGWIRQPPGKGLEWIGSIYYSGSTYYNPSLRSRVTISLDTSMNQFSLKLSSVTAADTAVYYCAEEYYDFWRTQDWGQGTLVTVSS

SEQ ID NO: 50 - protein, 10 aa, Homo sapiens
GGSITSSPYY

SEQ ID NO: 51 - protein, 7 aa, Homo sapiens
IYYSGST

SEQ ID NO: 52 - protein, 12 aa, Homo sapiens
AEEYYDFWRTQD

SEQ ID NO: 53 - protein, 107 aa, Homo sapiens
EIVLTQSPGTLSLSPGERATISCRASQSVSGRNLAWYQQKPGQAPRLLIYAASNRATGIPDRFSGSGSGTDFTLTISRLEPEDFAVYYCQQYGTSKTFGQGTKVEIK

SEQ ID NO: 54 - protein, 7 aa, Homo sapiens
QSVSGRN

SEQ ID NO: 55 - skipped

SEQ ID NO: 56 - protein, 8 aa, Homo sapiens
QQYGTSKT

SEQ ID NO: 57 - protein, 124 aa, Homo sapiens
QVQLVQSGVEVKKPGASVKVSCKASGYTFTRHGITWVRQAPGQGLEWMGWIAPYNGDTIYAEKFQARVTLTMDTSTNTVYLEMRSLSSDDTAVYYCGRDSVEEAFPISYYFDYWGQGTLVTVSS

SEQ ID NO: 58 - protein, 8 aa, Homo sapiens
GYTFTRHG

SEQ ID NO: 59 - protein, 8 aa, Homo sapiens
IAPYNGDT

SEQ ID NO: 60 - protein, 17 aa, Homo sapiens
GRDSVEEAFPISYYFDY

SEQ ID NO: 61 - protein, 110 aa, Homo sapiens
QSVLTQPRSVSGSPGQSVTISCTGTTNEIGIYNFVSWYQHHPGEAPRLIMYDVSKRPSGVPDRFSGSKSGNTASLTISGLQTEDEAAYYCCSYVGSYKLLFGGGTKLTVL

SEQ ID NO: 62 - protein, 9 aa, Homo sapiens
TNEIGIYNF

SEQ ID NO: 63 - skipped

SEQ ID NO: 64 - protein, 10 aa, Homo sapiens
CSYVGSYKLL

SEQ ID NO: 65 - protein, 466 aa, synthetic construct
MNPNQKIITIGSVLIIATICFLMQIAILVTTVTLHFKQHDYNSPPNNQAMLCEPTIIERNTTEIVYLTNTTIEKEICPKLAEYRNWSKPQITGFAPFSKDNSIRLSAGGDIWVTREPYVSCDPDKCYQFALGQGTTLNNGHSNNTVHDRTPYRTLLMNELGVPFHLGTRQVCMAWSSSSCHDGKAWLHVCITGNDNNATASFIYNGRLVDSIGSWSKNILRTQESECVCINGTCTVVMTDGSASGKADTKILFVEEGKIVHISTLSGSAQHVEECSCYPRFPGVRCVCRDNWKGSNRPIVDINVKNYSIVSSYVCSGLVGDTPRKSDSVSSSYCLDPNNEKGGHGVKGWAFDDGNDVWMGRTINETLRLGYETFKVIEGWSKANSKLQTNRQVIVEKGDRSGYSGIFSVEGKSCINRCFYVELIRGRKEETKVWWTSNSIVVFCGTSGTYGTGSWPDGADINLMPI

SEQ ID NO: 66 - protein, 467 aa, synthetic construct
MNPNQKIITIGSVLTISTICFFMQIAILITTVTLHFKQYEFNSPPNNQVMLCEPTIIERNITEIVYLTNTTIEKEICPKPAEYRNWSKPQITGFAPFSKDNSIRLSAGGDIWVTREPYVSCDLDKCYQFALGQGTTLNNVHSNNTVRDRTPYRTLLMNELGVPFHLGTRQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLDPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADILSLMHI